(19) United States
(12) Patent Application Publication
(10) Pub. No.: US 2017/0183742 A1
(43) Pub. Date: Jun. 29, 2017

(54) METHODS FOR PREDICTING THE SURVIVAL TIME OF PATIENTS SUFFERING FROM CANCER

(71) Applicants: INSERM (INSTITUT NATIONAL DE LA SANTÉ ET DE LA RECHERCHE MÉDICALE), Paris (FR); INSTITUT RÉGIONAL DU CANCER DE MONTPELLIER, Montpellier (FR); UNIVERSITE DE MONTPELLIER, Montpellier (FR)

(72) Inventors: Alain THIERRY, Montpellier (FR); Safia MESSAOUDI, Montpellier (FR); Florent MOULIERE, Montpellier (FR)

(73) Assignees: INSERM (INSTITUT NATIONAL DE LA SANTÉ ET DE LA RECHERCHE MÉDICALE), Paris (FR); INSTITUT RÉGIONAL DU CANCER DE MONTPELLIER, Montpellier (FR); UNIVERSITE DE MONTPELLIER, Montpellier (FR)

(21) Appl. No.: 15/314,010

(22) PCT Filed: May 27, 2015
(86) PCT No.: PCT/EP2015/061667

§ 371 Date: Nov. 25, 2016

(30) Foreign Application Priority Data
May 27, 2014 (EP) 14305793.3

(51) Int. Cl. C12Q 1/68

(52) U.S. Cl. CPC C12Q 1/6886; C12Q 2600/112; C12Q 2600/118; C12Q 2600/156

(57) ABSTRACT

The present invention relates to methods for predicting the survival time of patients suffering from cancer. Said methods are based on the quantification and analysis of the cell free nucleic acids that are present in a sample from the patient and typically include the determination of the level of the mutant nucleic acid which contains a mutation of interest, the calculation of the mutation load for said mutation of interest, the calculation of the DNA integrity index or a combination thereof.

### FIELD OF THE INVENTION

The present invention relates to methods for predicting the survival time of patients suffering from cancer.

### BACKGROUND OF THE INVENTION

Colorectal cancer (CRC) is the third most common cancer with nearly 1.4 million new cases in 2012 and 600,000 deaths per year (1). There is a strong need of a non-invasive tool to improve the prognosis evaluation for CRC patients, particularly for patients in early stage but there is also an urgent need to stratify the stage IV patients (2). Indeed, 25% of CRC patients are at the metastatic stage when CRC is diagnosed. Current prognostic gold standard for CRC patient classification remains the TNM classification (2). In metastatic CRC patients (mCRC), it is known that there is a wide diversity of outcome and there is no specific prognostic validated biomarker for the management of mCRC. However, Carcinogenic Embryonic Antigen (CEA) level is currently measured at the diagnosis time to establish a prognostic of the disease and constitutes a tool for the follow-up of the disease. Nevertheless, CEA is not specific to colorectal tumor and not specific to tumor process, and today, it is urgent to find a colorectal tumor specific biomarker.

Circulating cell-free DNA (ccfDNA) is a valuable source of tumour material available with a simple blood sampling enabling a non-invasive quantitative and qualitative analysis of the tumour genome. ccfDNA is released by tumour cells and exhibits the genetic and epigenetic alterations of the tumour of origin (3). The clinical significance of tumor-derived ccfDNA released in the blood of patients with colorectal cancer has already been investigated as a prognosis tool in previous studies with various technological approaches (4-6). In a recent large meta-analysis, a marked correlation between ccfDNA concentration and survival for metastatic CRC patients has been observed, and patients with relatively low levels of ccfDNA lived significantly longer than patients with higher levels (7). Prognosis relevance of ccfDNA levels in other cancer types has also been detailed for advanced breast cancer (8), lung cancer (9), prostate cancer (10) and other cancer types (11). Epigenetic alterations on ccfDNA have also been studied as a potential biomarker for CRC prognosis (12; 13).

However the majority of these studies are focusing on the concentration of total ccfDNA in the blood or on the detection of genetic or epigenetic alteration (14). However relation between total ccfDNA concentration and outcome may be biased since an increase in the level of total cfDNA might also be indicative of non-cancerous disease (inflammation, trauma) (15). The limited specificity of this quantitative estimation of the total level of ccfDNA leads to estimate also the qualitative alterations in ccfDNA and the fragmentation level of ccfDNA. Multi-marker analysis on melanoma patients seems an interesting approach for improving the utilization of ccfDNA as a prognosis tool (16). Modification in the DNA integrity index (ratio of long DNA fragments on short DNA fragments) indicating a greater integrity of the ccfDNA (17), or a reduction in this integrity, has been also investigated as a predictive tool for cancer progression.

The inventors were the first to demonstrate that tumor-derived circulating DNA was highly fragmented and mainly composed of <100 bp fragments by Q-PCR and AFM (18-21) which is smaller than the observed size between 145 and 180 bp reported in the literature (2, 14). Based upon this discovery, they designed Intplex, an allele specific Q-PCR based system targeting short sequences of DNA specifically adapted for ccfDNA analysis. With this specific and adapted design, they confirmed the powerful biomedical potential of ccfDNA analysis: The inventors showed the high diagnostic potential of ccfDNA concentration allowing discrimination between healthy subjects and cancer patients (20), they validated the detection of KRAS/BRAF point mutation in a cohort of 106 clinical samples from mCRC patients (22) with 98% of specificity with tumor-tissue analysis in a blinded clinical study. This work followed the standards for reporting diagnostic accuracy (STARD) guideline. Intplex allows the determination of the mutation load (mA %) which is the proportion of mutant ccfDNA in total ccfDNA reflecting the proportion of specific tumor ccfDNA in total ccfDNA. Targeting short sequences lead to find that up to 60% of total ccfDNA could be derived from the tumor (21) breaking the previous literature statement describing that tumor-derived ccfDNA was a tiny portion of total ccfDNA (23).

### SUMMARY OF THE INVENTION

The present invention relates to methods for predicting the survival time of patients suffering from cancer. Said methods are based on the quantification and analysis of the cell free nucleic acids that are present in a sample from the patient and typically include the determination of the level of the mutant nucleic acid which contains a mutation of interest, the calculation of the mutation load for said mutation of interest, the calculation of the DNA integrity index or a combination thereof. In particular, the present invention is defined by the claims.

### DETAILED DESCRIPTION OF THE INVENTION

The inventors have investigated with their Q-PCR multi-marker approach the overall survival of 106 metastatic colorectal cancer (mCRC) patients collected from three clinical centres. This is the biggest cohort of mCRC patients studied for potential prognostic interest of ccfDNA analysis. In all patients, the concentration of total ccfDNA, the determination of the main KRAS and BRAF mutations, the concentration of mutant ccfDNA, the proportion of mutation, and the integrity of ccfDNA were simultaneously determined for the first time. Each of these parameters was tested in univariate analysis for overall survival. Then the inventors have implemented these different parameters in a multi-marker analysis, and investigated if this multi-parametric analysis might improve the prognosis score for predicting patients overall survival in our study. Those results were compared to the prognostic value of CEA. The inventors show that the level of the mutant nucleic acids, the mutation load, and the DNA integrity index are correlated with the survival time of the patient.

### General Definitions

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the patient suffers from a colorectal cancer, more particularly a metastatic colorectal cancer.

The methods of the invention are particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely.

Typically, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

As used herein the term “nucleic acid” has its general meaning in the art and refers to refers to a coding or non coding nucleic sequence. Nucleic acids include DNA (deoxyribonucleic acid) and RNA (ribonucleic acid). Example of nucleic acid thus include but are not limited to DNA, mRNA, tRNA, rRNA, tmRNA, miRNA, piRNA, snoRNA, and snRNA. Typically, the nucleic acid according to the invention has a length of at 20 base pairs. According to the invention, the nucleic acid may originate form the nucleus of the cancer cells. By “cell free nucleic acid” it is meant that the nucleic acid is released by the cell and present in the sample. In some embodiments, the cell free nucleic acid is circulating cell-free DNA (ccfDNA).

As used herein the term “sample” refers to any biological sample obtained from the patient that is liable to contain cell free nucleic acids. Typically, samples include but are not limited to body fluid samples, such as blood, ascite, urine, amniotic fluid, feces, saliva or cerebrospinal fluids. In some embodiments, the sample is a blood sample. By “blood sample” it is meant a volume of whole blood or fraction thereof, e.g., serum, plasma, etc. Any methods well known in the art may be used by the skilled artisan in the art for extracting the free cell nucleic acid from the prepared sample. For example, the method described in the EXAMPLE may be used.

As used herein, the term “primer” refers to an oligonucleotide, whether occurring naturally as in a purified restriction digest or produced synthetically, which is capable of acting as a point of initiation of nucleic acid sequence synthesis when placed under conditions in which synthesis of a primer extension product which is complementary to a nucleic acid strand is induced, i.e. in the presence of different nucleotide triphosphates and a polymerase in an appropriate buffer (“buffer” includes pH, ionic strength, cofactors etc.) and at a suitable temperature. Typically, a primer has a length of 10; 11; 12; 13; 14; 15; 16; 17; 18; 19; 20; 21; 22; 23; 24; 25; 26; 27; 28; 29; or 30 nucleotides. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. Primers are typically labelled with a detectable molecule or substance, such as a fluorescent molecule, a radioactive molecule or any others labels known in the art. Labels are known in the art that generally provide (either directly or indirectly) a signal. The term “labelled” is intended to encompass direct labelling of the probe and primers by coupling (i.e., physically linking) a detectable substance as well as indirect labeling by reactivity with another reagent that is directly labeled. Examples of detectable substances include but are not limited to radioactive agents or a fluorophore (e.g. fluorescein isothiocyanate (FITC) or phycoerythrin (PE) or Indocyanine (Cy5)).

### Methods (A) Based on the Level of the Mutant Nucleic Acids

An object of the present invention to a method (A) for predicting the survival time of a patient suffering from a cancer comprising the steps of i) extracting the cell free nucleic acids from a sample obtained from the patient, ii) determining the level of the mutant nucleic acids liable to be present in the extracted cell free nucleic acids, iii) comparing the level determined at step ii) with a predetermined reference value and iv) concluding that the patient will a short survival time when the level determined at step ii) is higher than the predetermined reference value or concluding that the patient will have a long survival time when the level determined at step ii) is lower than the predetermined reference value.

As used herein the term “mutant nucleic acid” refers to a nucleic acid bearing a point mutation of interest. Cell free nucleic acid in a patient suffering from a cancer is constituted of nucleic acids of tumor and non-tumor origin. According to the invention, it is thus important to select a mutation which has a tumor origin to quantify only the nucleic acids which derives from cancer cells. In some embodiments, the mutation directly contributes to the initiation of the malignant transformation (“driver mutation”). In some embodiments, the mutation is located in a gene selected from the group consisting of KRAS, BRAF, NRAS, TP53, APC, MSH6, NF1, PIK3CA, SMAD4, EGFR, CDKN2A, IDH1, PTEN, SMARCB1, CTNNB1, HNF1A, VHL, ATM, EZH2, RET, NRAS, PTCH1, KIT, NF2, PDGFRA, PPP2R1A, STK11, MLL3, FOXL2, GNAS, HRAS, FGFR3, PTCH1, and CDH1. For example, the mutation is located in a gene selected from the group consisting of TP53 (394, 395, 451, 453, 455, 469, 517, 524, 527, 530, 586, 590, 637, 641, 724, 733, 734, 743, 744, 817, 818, 819, 820, 839, 844, 916), APC (2626, 3340, 3907, 3934, 3964, 4012, 4099, 4132, 4133, 4285, 4286, 4348, 4729), MSH6 (1168), NF1 (3827, 3826), PIK3CA (1530, 1624, 1633, 1634, 1636, 1656, 3140, 3140, 3140), SMAD4 (502, 931, 932, 988, 989, 1051, 1082, 1156, 1332, 1333, 1519, 1596, 1597, 1598, 1606), EGFR (2155, 2155, 2156, 2303, 2369, 2573; deletions/loss (2230 to 2244, from 2308 à 2328), CDKN2A (172, 205, 238, 239, 298, 250, 322, 369, 427, 394), IDH1 (394; 395), PTEN (125, 126, 182, 302, 314, 387, 388, 389, 1911, 577, 518, 519, 697, 698, 1003, 1004), SMARCB1 (118, 153, 154, 379, 380, 425, 471, 472, 473, 601, 618, 619, 777, 776, 778), CTNNB1 (7, 94, 95, 98, 100, 101, 110, 121, 122, 133, 134, 170), HNF1A (82, 81, 83, 196, 378, 379, 493, 494, 495, 526, 527, 617, 618, 685, 710, 749, 787, 817), VHL (194, 203, 241, 266, 340, 343, 388, 452, 473, 480, 478), ATM (1229, 1810, 2571, 2572, 2573, 3925, 8774, 9023), EZH2 (1936, 1937), RET (2753), NRAS (181, 182, 183), PTCH1 (135, 338, 416, 417, 1242, 1243, 1244, 1280 1281, 1284, 1301, 1302, 1315), KIT (1668, 1669, 1670, 1679, 1680, 1681, 1682, 1727, 1728, 1924, 1925, 1961, 1962, 2467, Deletions from 1645 à 1727), NF2 (168, 169, 170, 459, 460, 586, 592, 634, 655, 656, 784, 1021, 1022, 1396, PDGFRA (1680, 1681, 1682, 1975, 1976, 1977), MEN1 (124, 256, 291, 292, 293), PPP2R1A (536, 767), STK11 (196, 910), MLL3 (1097, 4432, 6301, 6851, 8911, 10040, 10495, 12048, 12165), FOXL2 (402), GNAS (601, 602, 680), HRAS (34, 35, 36, 37, 39, 181, 182), FGFR3 (742, 743, 744, 746, 1108, 1111, 1112, 1113, 1114, 1115, 1116, 1117, 1118, 1949), PTCH1 (549, 550, 584, 1093, 1249, 1804, 2446, 3054, 3944, 3945, 3946), and CDH1 (367, 368, 1000, 1057, 1108, 1204, 1436, 1437, 1742) (wherein for each gene the position number of the hot spot mutation in the cDNA rare indicated upon NCBI 36: Ensembl Contig view <http://may2009.archive.ensembl.org/Homo_sapiens/Location/). In some embodiments, the mutation is a KRAS mutation. The term “KRAS mutation” includes any one or more mutations in the KRAS (which can also be referred to as KRAS2 or RASK2) gene. For example, the KRAS mutations are located in exon 3 or exon 4 of the gene. Examples of KRAS mutations include, but are not limited to, G12C, G12D, G13D, G12R, G12S, and G12V. In some embodiments, the mutation is a BRAF mutation. The term “BRAF mutation” includes any one or more mutations in the BRAF (which can also be referred to as serine/threonine-protein kinase B-Raf or B-Raf) gene. Typically, the BRAF mutation is V600E.

Determination of the level of the nucleic acid can be performed by a variety of techniques well known in the art. Advantageously, the analysis of the expression level of a nucleic acid involves the process of nucleic acid amplification, e. g., by Q-PCR,ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 57, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854,033) or any other nucleic acid amplification method, followed by the detection of the amplified molecules using techniques well known to those of skill in the art. Q-PCR is the preferred method.

Typically the primers are thus designed to amplify a target nucleic acid sequence having less than 100 base pairs and which comprises the mutation of interest. Typically, the target nucleic acid sequence has a length inferior to 110 base pairs. In some embodiments, the target nucleic acid sequence for determining the level of the mutant nucleic acids has length of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; or 110 base pairs.

Examples of primers that can be used in the present invention are described in the EXAMPLE.

In some embodiments, the method of is performed for at least 2 mutations. In some embodiments, the method of the invention is performed with 2, 3, 4, 5 or n mutations (i.e. n is an integer number). In some embodiments, the mutations are located in different genes (e.g. KRAS and BRAF genes). In some embodiments, the mutations are located in the same genes. In some embodiments, the mutations are located in the same exon of the same gene. In some embodiments, the mutations are located in different exons of the same gene. For each mutation (M)ₙ the level of the mutant nucleic acids (ELM)ₙ is determined and compared with its corresponding predetermined reference value (ELRM)ₙ. The higher the number of (ELM)ₙ are higher than their corresponding predetermined values (ELRM)ₙ, the shorter will be the survival time of the patient.

### Methods (B) Based on the Calculated Mutation Load

A further object of the present invention relates to a method (B) for predicting the survival time of a patient suffering from a cancer comprising the steps of i) extracting the cell free nucleic acids from a sample obtained from the patient, ii) determining the level of the mutant nucleic acids liable to be present in the extracted cell free nucleic acids, iii) determining the total concentration of cell free nucleic acids, iv) calculating the ratio of the level determined at step ii) to the concentration determined at step iii), v) comparing ratio determined at step iv) with a predetermined reference value and vi) concluding that the patient will a short survival time when the ratio determined at step iv) is higher than the predetermined reference value or concluding that the patient will have a long survival time when the level determined at step iv) is lower than the predetermined reference value.

The term “mutant nucleic acid” has the same meaning as defined above. Accordingly methods for quantifying the mutant nucleic acid are the same.

Methods for determining the total concentration of cell free nucleic acids are well known in the art. For example, the method is described in WO2012/028746. Q-PCR is thus the preferred method for determining said concentration. In some embodiment, the method consists of amplifying and quantifying a target acid nucleic sequence which has about the same size than the target nucleic acid sequence used for quantifying the mutant nucleic acid sequence. Typically, the length of the target nucleic acid sequence for determining the total concentration is 1; 2; 3; 4; 5; 6; 7; 8; 9; 10; 11; 12; 13; 14; or 15% longer or shorter than the target nucleic acid sequence selected for determining the level of the mutant nucleic acid. Accordingly, the target nucleic acid sequence for determining the total concentration of the cell free nucleic acid has a length inferior to 110 base pairs. In some embodiments, the target nucleic acid sequence for determining the total concentration of cell free nucleic acids of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; or 110 base pairs. In some embodiments, the target nucleic sequence selected for determining the total concentration of cell free nucleic acids and the target nucleic acid sequence selected for determining the level of the mutant nucleic acids are located in the same gene (e.g KRAS gene or BRAF gene). In some embodiments, the target nucleic sequence selected for determining the total concentration of cell free nucleic acids and the target nucleic acid sequence selected for determining the level of the mutant nucleic acids are located in the same exon.

According to the invention, the ratio of the level determined at step ii) to the level determined at step iii) is typically named as the “mutation load”.

In some embodiments, the method is performed for at least 2 mutations. In some embodiments, the method of the invention is performed with 2, 3, 4, 5 or n mutations (i.e. n is an integer number). For each mutation (M)ₙ the mutation load (ML)ₙ is determined and compared with its corresponding predetermined reference value (MLR)ₙ. The higher the number of (ML)ₙ are higher than their corresponding predetermined values (MLR)ₙ, the shorter will be the survival time of the patient.

### Methods (C) Based on the DNA Integrity Index

A further object of the present invention relates to a method (C) for predicting the survival time of a patient suffering from a cancer comprising the steps of i) extracting the cell free nucleic acids from a sample obtained from the patient, ii) determining the level of the nucleic acids having a length inferior to 110 base pairs, iii) determining the level of the nucleic acids having a length superior to 250 base pairs, iv) calculating the ratio of the level determined at step iii) to the level determined at step ii), v) comparing the ratio determined at step iv) with a predetermined reference value and vi) concluding that the patient will a short survival time when the ratio determined at step iv) is lower than the predetermined reference value or concluding that the patient will have a long survival time when the level determined at step iv) is higher than the predetermined reference value.

Once again, Q-PCR is the preferred method for determining the level of the nucleic acids having a length inferior to 110 base pairs and the level of the nucleic acids having a length of at least 250 base pairs (e.g. see the method is described in WO2012/028746). In some embodiment, the method consists of amplifying and quantifying a first target acid nucleic sequence having a length of inferior to 110 base pairs and a second target acid nucleic sequence having a length of at least 250 base pairs. In some embodiments, the first target nucleic acid sequence has a length of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; or 110 base pairs. In some embodiments, the second target nucleic acid sequence has a length of 250; 251; 252; 253; 254; 255; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350 base pairs. In some embodiments, the first and second target nucleic sequences are located in the same gene (e.g KRAS gene or BRAF gene). In some embodiments, the first and second target nucleic sequences are located in the same exon. In some embodiments, the first and second target nucleic sequences allow the amplification and quantification of nucleic acids having the same mutation of interest (i.e. as above described).

According to the invention the ratio of the level determined at step iii) to the level determined at step ii) is typically named the “DNA Integrity Index” or “DII”.

In some embodiments, the method is performed for at least 2 mutations. In some embodiments, the method of the invention is performed with 2, 3, 4, 5 or n mutations (i.e. n is an integer number). For example, when the index is determined for a KRAS mutation it is named the “KRAS DII” and when the index is determined for a BRAF mutation, it is named the “BRAF DII”. For each mutation (M)ₙ the DNA integrity Index (DII)ₙ is determined and compared with its corresponding predetermined reference value (DIIR)ₙ. The higher the number of (DII)ₙ are lower than their corresponding predetermined values (DIIR)ₙ, the shorter will be the survival time of the patient.

### Combination Methods

A further objection the methods as above described (A, B or C) may be combined with any method well known in the art. In some embodiments, the method A, B or C is combined with the determination the total concentration of cell free nucleic acids present in the sample. In some embodiments, when no mutation (e.g. driver mutation) could be determined, it is suitable to combine method (C) (DNA Integrity Index) with the method which consists of determining the total concentration of cell free nucleic acids present in the sample. Accordingly, in some embodiments, the present invention relates to a method for predicting the survival time of a patient suffering from a cancer comprising the steps of i) extracting the cell free nucleic acids from a sample obtained from the patient, ii) determining the total concentration of cell free nucleic acids present in the sample, iii) determining the level of the nucleic acids having a length inferior to 110 base pairs, iv) determining the level of the nucleic acids having a length of superior to 250 base pairs, v) calculating the ratio of the level determined at step iv) to the level determined at step iii), vi) comparing the total concentration of cell free nucleic acids with its corresponding predetermined reference value, vii) comparing the ratio determined at step v) with its corresponding predetermined reference value and viii) concluding that the patient will a short survival time when
the total concentration determined at step i) is higher that its corresponding reference value and
the ratio determined at step v) is lower than its corresponding predetermined reference value.

A further object of the present invention relates to a method which combines at least two methods as above described (i.e. A, B or C). In some embodiment, the present invention relates to a method which combines method (A) and method (B). In some embodiments, the present invention relates to a method which combines method (A) and method (C). In some embodiments, the present invention relates to a method which combines method (B) and method (C). In some embodiment, the present invention relates to a method which combines method (A), method (B) and method (C).

A further object of the present invention relates to a method for predicting the survival time of a patient suffering from a cancer which combines in a single assay performed in a sample obtained from the patient, the detection of a mutation of interest, the determination of the level of the mutant nucleic acid which contains the mutation of interest, the calculation of the mutation load as defined above for said mutation of interest, the calculation of the DNA integrity index as defined above for said mutation of interest and the determination of the total concentration of the cell free nucleic acid present in the sample. This method thus implements the 3 above described method. Typically, the single multi-marker assay is Intplex® as described in WO2012/028746 and Mouliere F et al, Multi-marker analysis of circulating cell-free DNA toward personalized medicine for colorectal cancer. Mol Oncol. 2014 March 24. Briefly, Intplex® is based on a nested diagram, where two short amplicons (60-100 bp±10 bp) were implemented among a larger amplicon (300±bp). One of the short amplicon was targeting a specific locus hotspot of interest (e.g. a KRAS mutation or a BRAF mutation). The other short amplicon was designed for amplifying a WT sequence, a sequence which does not bear the mutation of interest. Primer design and validation of said pimeres are typically performed as previously described in Thierry A R et al, Clinical validation of the detection of KRAS and BRAF mutations from circulating tumor DNA. Nat Med. 2014 April; 20(4):430-5.

Accordingly, in some embodiments, the method of the present invention comprises the step consisting of i) extracting the cell free nucleic acids from a sample obtained from the patient, ii) determining the level of the mutant nucleic acids (as above described), iii) determining the total concentration of cell free nucleic acids present in the sample, iv) determining the mutation load (as above described), v) calculating the DNA integrity index, (as above described), vi) comparing the level of the mutant nucleic acids with its corresponding predetermining reference value, vii) comparing the total concentration of cell free nucleic acids with its corresponding predetermined reference value viii) comparing the mutation load with its corresponding predetermined reference value, ix) comparing the DNA integrity index with is corresponding predetermined reference value and x) finally concluding that the patient will a short survival time when
the mutation is detected
the level of the mutant nucleic acids is higher than its corresponding predetermined reference value
the total concentration of cell free nucleic acids is higher than its corresponding predetermined reference value
the mutation load is higher than its corresponding predetermined reference value
the DNA integrity index is lower than its corresponding reference value.

### Predetermined Reference Values

Typically, the predetermined reference value can be relative to a number or value derived from population studies, including without limitation, patients of the same or similar age range, patients in the same or similar ethnic group, and patients having the same severity of cancer. Such predetermined reference values can be derived from statistical analyses and/or risk prediction data of populations obtained from mathematical algorithms and computed indices of the disease.

Typically, the predetermined reference value is a threshold value or a cut-off value. A “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the expression level of the marker of interest (e.g. level of the mutant nucleic acids, mutation load, DII, or total concentration of cell free nucleic acids) in properly banked historical patient samples may be used in establishing the predetermined reference value.

In some embodiments, the predetermined reference value is the median measured in the population of the patients for the marker of interest (e.g. level of the mutant nucleic acids, mutation load, DII, or total concentration of cell free nucleic acids).

In some embodiments, the threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the marker of interest (e.g. level of the mutant nucleic acids, mutation load, DII, or total concentration of cell free nucleic acids) in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator the reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is typically determined by carrying out a method comprising the steps of:

a) providing a collection of blood samples from patient suffering from the same cancer;

b) providing, for each blood sample provided at step a), information relating to the actual clinical outcome for the corresponding patient (i.e. the duration of the disease-free survival (DFS) and/or the overall survival (OS));

c) providing a serial of arbitrary quantification values;

d) determining the level of the marker of interest (e.g. level of the mutant nucleic acids, mutation load, DII, or total concentration of cell free nucleic acids) for each blood sample contained in the collection provided at step a);

e) classifying said blood samples in two groups for one specific arbitrary quantification value provided at step c), respectively: (i) a first group comprising blood samples that exhibit a quantification value for level that is lower than the said arbitrary quantification value contained in the said serial of quantification values; (ii) a second group comprising blood samples that exhibit a quantification value for said level that is higher than the said arbitrary quantification value contained in the said serial of quantification values; whereby two groups of blood samples are obtained for the said specific quantification value, wherein the blood samples of each group are separately enumerated;

f) calculating the statistical significance between (i) the quantification value obtained at step e) and (ii) the actual clinical outcome of the patients from which blood samples contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant) has been calculated at step g).

For example the level of the marker of interest (e.g. level of the mutant nucleic acids, mutation load, DII, or total concentration of cell free nucleic acids) has been assessed for 100 blood samples of 100 patients. The 100 samples are ranked according to the level of the marker of interest (e.g. level of the mutant nucleic acids, mutation load, DII, or total concentration of cell free nucleic acids). Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the level of the marker of interest (e.g. level of the mutant nucleic acids, mutation load, DII, or total concentration of cell free nucleic acids) corresponding to the boundary between both subsets for which the p value is minimum is considered as the predetermined reference value. It should be noted that the predetermined reference value is not necessarily the median value of levels of the marker of interest (e.g. level of the mutant nucleic acids, mutation load, DII, or total concentration of cell free nucleic acids). Thus in some embodiments, the predetermined reference value thus allows discrimination between a poor and a good prognosis with respect to DFS and OS for a patient. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the level of the marker of interest (e.g. level of the mutant nucleic acids, mutation load, DII, or total concentration of cell free nucleic acids) with the range of values which are identified. In certain embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. Therefore, a patient may be assessed by comparing values obtained by measuring the level of the marker of interest (e.g. level of the mutant nucleic acids, mutation load, DII, or total concentration of cell free nucleic acids), where values greater than 5 reveal an increased risk of having a poor prognosis and values less than 5 reveal a decreased risk of a poor prognosis. In some embodiments, a patient may be assessed by comparing values obtained by measuring the level of the marker of interest (e.g. level of the mutant nucleic acids, mutation load, DII, or total concentration of cell free nucleic acids) and comparing the values on a scale, where values above the range of 4-6 indicate an increased risk having a poor prognosis and values below the range of 4-6 indicate a decreased risk of having a poor prognosis, with values falling within the range of 4-6 indicating an intermediate prognosis.

### Quantitative PCR (OPCR)

The template nucleic acid need not be purified. Nucleic acids may be extracted from a sample by routine techniques such as those described in Diagnostic Molecular Microbiology: Principles and Applications (Persing et al. (eds), 1993, American Society for Microbiology, Washington D.C.).

U.S. Pat. Nos. 4,683,202, 4,683,195, 4,800,159, and 4,965,188 disclose conventional PCR techniques. PCR typically employs two oligonucleotide primers that bind to a selected target nucleic acid sequence. Primers useful in the present invention include oligonucleotides capable of acting as a point of initiation of nucleic acid synthesis within the target nucleic acid sequence. A primer can be purified from a restriction digest by conventional methods, or it can be produced synthetically. If the template nucleic acid is double-stranded (e.g. DNA), it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min). If the double-stranded template nucleic acid is denatured by heat, the reaction mixture is allowed to cool to a temperature that promotes annealing of each primer to its target sequence on the target nucleic acid sequence. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min).

QPCR involves use of a thermostable polymerase. The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus fiavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished. Typically, the polymerase is a Taq polymerase (i.e. Thermus aquaticus polymerase).

The primers are combined with PCR reagents under reaction conditions that induce primer extension. Typically, chain extension reactions generally include 50 mM KCl, 10 mM Tris-HCl (pH 8.3), 15 mM MgCl2, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target nucleic acid sequence molecule. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

Quantitative PCR is typically carried out in a thermal cycler with the capacity to illuminate each sample with a beam of light of a specified wavelength and detect the fluorescence emitted by the excited fluorophore. The thermal cycler is also able to rapidly heat and chill samples, thereby taking advantage of the physicochemical properties of the nucleic acids and thermal polymerase.

In order to detect and measure the amount of amplicon (i.e. amplified target nucleic acid sequence) in the sample, a measurable signal has to be generated, which is proportional to the amount of amplified product. All current detection systems use fluorescent technologies. Some of them are non-specific techniques, and consequently only allow the detection of one target at a time. Alternatively, specific detection chemistries can distinguish between non-specific amplification and target amplification. These specific techniques can be used to multiplex the assay, i.e. detecting several different targets in the same assay.

SYBR® Green I:

SYBR® Green I is the most commonly used dye for non-specific detection. It is a double-stranded DNA intercalating dye, that fluoresces once bound to the DNA. A pair of specific primers is required to amplify the target with this chemistry. The amount of dye incorporated is proportional to the amount of generated target. The dye emits at 520 nm and fluorescence emitted can be detected and related to the amount of target. The inconvenience of this technique is that the SYBR® Green I will bind to any amplified dsDNA. Consequently, primer dimers or unspecific products introduce a bias in the quantification. However, it is still possible to check for the specificity of the system by running a meltcurve at the end of the PCR run. The principle is that every product has a different dissociation temperature, depending of the size and base contents, so it is still possible to check the number of products amplified. A valid SYBR® assay—primer pair—should produce a unique, well defined peak on the meltcurve. For these reasons, SYBR® Green I is rarely used for qualitative PCR. However, SYBR® Green I is often used as the first step to optimize a specific detection system assay, to check the specificity of the primers and validate the design.

High Resolution Melting Dyes (HRM Dyes):

High Resolution Meltcurve analysis is a newly emerging technology, which characterizes nucleic acid samples based on their dissociation behaviour. It combines the principle of intercalating dyes, meltcurve analyses and the application of specific statistical analyses. HRM uses the fundamental property of the separation of the two strands of DNA with heat (melting), and the monitoring of this melting with a fluorescent dye. On the contrary of SYBR Green, HRM dyes do not inhibit PCR at high concentration. The dye can consequently saturate the amplified target dsDNA and fluoresces. Melting temperature of a dsDNA target depends on GC content, length, and sequence. Due to the high sensitivity of HRM dyes, even a single base change will induce differences in the melting curve, and consequently in fluorescence (Erali M. et al., 2008). This emerging method is less expensive and as precise than probe-based methods. Only a few thermocyclers on the market currently allow the use of this technology, among them the Roche LightCycler®480, the Corbett Life Science Rotor-Gene™ 6000, and the ABI Prism®7500. The main HRM dyes available are EvaGreen, LCGreen®, SYTO® 9 and BEBO.

TaqMan® Probes=Double-Dye Probes:

TaqMan® probes, also called Double-Dye Oligonucleotides, Double-Dye Probes, or Dual-Labelled probes, are the most widely used type of probes and are often the method of choice for scientists who have just started using Real-Time PCR. They were developed by Roche (Basel, Switzerland) and ABI (Foster City, USA) from an assay that originally used a radio-labelled probe (Holland et al. 1991), which consisted of a single-stranded probe sequence that was complementary to one of the strands of the amplicon. A fluorophore is attached to the 5′ end of the probe and a quencher to the 3′ end. The fluorophore is excited by the machine and passes its energy, via FRET (Fluorescence Resonance Energy Transfer) to the quencher. Traditionally the FRET pair has been FAM as the fluorophore and TAMRA as the quencher. In a well designed probe, FAM does not fluoresce as it passes its energy onto TAMRA. As TAMRA fluorescence is detected at a different wavelength to FAM, the background level of FAM is low. The probe binds to the amplicon during each annealing step of the PCR. When the Taq polymerase extends from the primer which is bound to the amplicon, it displaces the 5′ end of the probe, which is then degraded by the 5′-3′ exonuclease activity of the Taq polymerase. Cleavage continues until the remaining probe melts off the amplicon. This process releases the fluorophore and quencher into solution, spatially separating them (compared to when they were held together by the probe). This leads to an irreversible increase in fluorescence from the FAM and a decrease in the TAMRA.

LNA® Double-Dye Probes:

LNA® (Locked Nucleic Acid) was developed by Exiqon® (Vedbaek, Denmark). LNA® changes the conformation of the helix and increases the stability of the duplex. The integration of LNA® bases into Double-Dye Oligonucleotide probes, opens up great opportunities to improve techniques requiring high affinity probes as specific as possible, like SNP detection, expression profiling and in situ hybridization. LNA® is a bicyclic RNA analogue, in which the ribose moiety in the sugar-phosphate backbone is structurally constrained by a methylene bridge between the 2′-oxygen and the 4′-carbon atoms. The integration of LNA® bases into probes changes the conformation of the double helix from the B to A type (Ivanova A. et al., 2007). LNA® conformation allows a much better stacking and therefore a higher stability. By increasing the stability of the duplex, the integration of LNA® monomers into the oligonucleotide sequence allows an increase of the melting Temperature (Tm) of the duplex. It is therefore possible to reduce the size of the probe, which increases the specificity of the probe and helps designing it (Karkare S. et al., 2006).

Molecular Beacon Probes:

Molecular Beacons are probes that contain a stem-loop structure, with a fluorophore and a quencher at their 5′ and 3′ ends, respectively. The stem is usually 6 bases long, should mainly consist of C's and G's, and holds the probe in the hairpin configuration (Li Y. et al., 2008). The ‘stem’ sequence keeps the fluorophore and the quencher in close vicinity, but only in the absence of a sequence complementary to the ‘loop’ sequence. As long as the fluorophore and the quencher are in close proximity, the quencher absorbs any photons emitted by the fluorophore. This phenomenon is called collisional (or proximal) quenching. In the presence of a complementary sequence, the Beacon unfolds and hybridizes to the target, the fluorophore is then displaced from the quencher, so that it can no longer absorb the photons emitted by the fluorophore, and the probe starts to fluoresce. The amount of signal is proportional to the amount of target sequence, and is measured in real time to allow quantification of the amount of target sequence (Takacs T. et al., 2008). The increase in fluorescence that occurs is reversible, (unlike TaqMan® probes), as there is no cleavage of the probe, that can close back into the hairpin structure at low temperature. The stem structure adds specificity to this type of probe, because the hybrid formed between the probe and target has to be stronger than the intramolecular stem association. Good design of Molecular Beacons can give good results, however the signal can be poor, as no physical separation of fluorophore from quencher occurs. Wavelength-Shifting Molecular Beacons are brighter than standard Molecular Beacons due to an enhanced fluorescence intensity of the emitter fluorophore. These probes contain a harvester fluorophore that absorbs strongly in the wavelength range of the monochromatic light source, an emitter fluorophore of the desired emission color, and a non-fluorescent (dark) quencher. In the absence of complementary nucleic acid targets, the probes are non-fluorescent, whereas in the presence of targets, they fluoresce, not in the emission range of the harvester fluorophore, that absorbs the light, but rather in the emission range of the emitter fluorophore. This shift in emission spectrum is due to the transfer of the absorbed energy from the harvester fluorophore to the emitter fluorophore by FRET, which only takes place in probes that are bound to the targets. Wavelength-Shifting Molecular Beacons are substantially brighter than conventional Molecular Beacons that cannot efficiently absorb energy from the available monochromatic light source (Tyagi S. et al., 2000).

Scorpions® Primers:

Scorpions® primers are suitable for both quantitative Real-Time PCR and genotyping/end-point analysis of specific DNA targets. They are PCR primers with a “stem-loop” tail consisting of a specific probe sequence, a fluorophore and a quencher. The “stem-loop” tail is separated from the PCR primer sequence by a “PCR blocker”, a chemical modification that prevents the Taq polymerase from copying the stem loop sequence of the Scorpions® primer. Such read-through would lead to non-specific opening of the loop, causing a non-specific fluorescent signal. The hairpin loop is linked to the 5′ end of a primer via a PCR blocker. After extension of the primer during PCR amplification, the specific probe sequence is able to bind to its complement within the same strand of DNA. This hybridization event opens the hairpin loop so that fluorescence is no longer quenched and an increase in signal is observed. Unimolecular probing is kinetically favorable and highly efficient. Covalent attachment of the probe to the target amplicon ensures that each probe has a target in the near vicinity. Enzymatic cleavage is not required, thereby reducing the time needed for signaling compared to TaqMan® probes, which must bind and be cleaved before an increase in fluorescence is observed. There are three types of Scorpions® primers. Standard Scorpions®, which consist of a bi-labelled probe with a fluorescent dye at the 5′ end and an internal non-fluorescent quencher. FRET Scorpions®, for use on a LightCycler® system. As the capillary system will only excite at 470 nm (FAM absorption wavelength) it is necessary to incorporate a FAM within the stem. A ROX is placed at the 5′end of the Scorpions® primer, FAM is excited and passes its energy onto the ROX. Duplex Scorpions® have also been developed to give much better signal intensity than the normal Scorpions® format. In Standard Scorpions® the quencher and fluorophore remain within the same strand of DNA and some quenching can occur even in the open form. In the Duplex Scorpions® the quencher is on a different oligonucleotide and physical separation between the quencher and fluorophore is greatly increased, reducing the quenching when the probe is bound to the target.

Hybridization Probes (Also Called FRET Probes):

Roche has developed hybridization probes (Caplin et al. 1999) for use with their LightCycler®. Two probes are designed to bind adjacent to one another on the amplicon. One has a 3′ label of FAM, whilst the other has a 5′ LC dye, LC red 640 or 705. When the probes are not bound to the target sequence, the fluorescent signal from the reporter dye is not detected. However, when the probes hybridize to the target sequence during the PCR annealing step, the close proximity of the two fluorophores allows energy transfer from the donor to the acceptor dye, resulting in a fluorescent signal that is detected.

TaqMan® MGB® Probes:

TaqMan® MGB® probes have been developed by Epoch Biosciences (Bothell, USA) and Applied Biosystems (Foster City, USA). They bind to the minor groove of the DNA helix with strong specificity and affinity. When the TaqMan® MGB® probe is complemented with DNA, it forms a very stable duplex with DNA. The probe carries the MGB® moiety at the 3′ end. The MGB strongly increases the probe Tm, allowing shorter, hence more specific designs. The probe performs particularly well with A/T rich regions, and is very successful for SNP detection (Walburger et al., 2001). It can also be a good alternative when trying to design a probe which should be located in the splice junction (for which conventional probes are hard to design). Smaller probes can be designed with Tm as 65-67° C., which gives a better discrimination (the probe is more specific for single mismatch). A good alternative to MGB probes are LNA® probes where the increase in Tm induced by the addition of LNA® bases is specific, contrary to the MGB moeity (cf. p. 15). During the primer extension step, the hybridized probe is cleaved by the 5′ exonuclease activity of Taq polymerase and an increase in fluorescence is seen. Fluorescence of the cleaved probe during PCR is monitored in Real-Time by the thermocycler.

MGB Eclipse® Probes:

MGB Eclipse® probes also known as QuantiProbes, have originally been developed by Epoch Biosciences (Bothell, USA). MGB Eclipse® probes carry a minor groove binder moiety that allows the use of short probes for very high specificity. These are short linear probes that have a minor groove binder and a quencher on the 5′ end and a fluorophore on the 3′end. This is the opposite orientation to TaqMan® MGB® probes and it is thought that the minor groove binder prevents the exonuclease activity of the Taq polymerase from cleaving the probe. The quencher is a Non Fluorescent Quencher also known as Eclipse Dark Quencher. Quenching occurs when the random coiling of the probe in the free form brings the quencher and the fluorophore close to another. The probe is straightened out when bound to its target and quenching is decreased, leading to an increase in fluorescent signal. The technologies that have been discussed above are the most widely used today, but numerous other technologies have occurred in publications, or are available on the market, such as: Resonsense probes, Light-up probes, HyBeacon® probes, LUX primers, Yin-yang probes, or Amplifluor®. You can contact us for more information on any of them.

The majority of the thermocyclers on the market now offer similar characteristics. Typically, thermocyclers involve a format of glass capillaries, plastics tubes, 96-well plates or 384-wells plates. The thermocylcer also involve a software analysis.

Typically quantitative PCR involves use of:
Taq polymerase: A HotStart Taq polymerase is inactive at low temperatures (room temperature). Heating at 95° C. for several—usually 5 to 10—minutes activates the enzyme, and the amplification can begin once the primers are annealed. The enzyme is not active until the entire DNA is denatured. Two major HotStart modifications exist, the antibody-blocked Taq and the chemically-blocked Taq. The antibody-blocked Taq is inactive because it is bound to a thermolabile inhibitor that is denatured during the initial step of PCR. The chemically-blocked Taq provides one clear advantage over the antibody-blocked Taq, as it is completely inactive at 60° C., (the hybridization temperature of primers), thus preventing the formation of non-specific amplification and reducing primer dimer formation.
dNTps/dUTps: Some kits contain a blend of dNTPs and dUTPs, other ones contain only dNTPs. Using only dNTPs increases the sensitivity, the reason being that the Taq incorporates more easily dNTPs than dUTPs. However, using a mix containing dUTPs brings security to the assay, in case of contamination from a previous PCR product. Thanks to the UNG activity in association with incorporated dUTPs, this contamination can be eliminated.
Uracil-N-Glycosylase: The Uracil-N-Glycosylase is an enzyme that hydrolyses all single-stranded and double-stranded DNA containing dUTPs. Consequently, if all PCR amplifications are performed in the presence of a dNTPs/dUTPs blend, by carrying a UNG step before every run it is possible to get rid of any previous PCR product.
ROX reference dye: Some thermocyclers require MasterMix containing ROX dye for normalization. This is the case for the ABI and Eppendorf machines, and optional on the Stratagene machines. If you work with such machines, it is easier to work with the ROX dye already incorporated in the MasterMix rather than adding it manually. It guarantees a higher level of reproducibility and homogeneity of your assays.
Fluorescein: For iCycler iQ®, My iQ® and iQ5 machines (BioRad thermocyclers), the normalization method for SYBR® Green assay uses Fluorescein to create a “virtual background”. As in the case for the ROX, it is better and easier to use a MasterMix that contains pre-diluted Fluorescein, guaranteeing higher reproducibility and homogeneity of your assays.
MgCl₂: MgCl₂ is necessary for the Taq activity. MgCl concentration in MasterMixes is optimized according to the amount of Taq and also the buffer composition. However, it may be necessary sometimes to add MgCl2 and most MasterMixes include an additional tube of MgCl2.
Inert colored dye: Some buffers also include an inert colored dye, to enable visualization of the buffer when loading in the wells. This colored dye has no effect on the sensitivity of the assay and is a convenient working tool. Note that such mixes, in combination with white plastic plates, provide better levels of fluorescence and a really easy way of working.

Well-designed primers and probes are a prerequisite for successful quantitative PCR. By using well-designed primers and probes, PCR efficiencies of 100% can be obtained. Typically primers are designed using a design software (for example Oligo® Primer Analysis Software). Most thermocycler softwares now offer tools to help in designing primers with the best characteristics. Some of the best softwares are Beacon Designer, Primer Express, and DNA Star . . . . Some other tools are freely available on the web, for example:
http://medgen.ugent.be/rtprimerdb/ (human primer and probe database)
http://frontend.bioinfo.rpi.edu/applications/mfold/ (for testing secondary structures)
http://www.ebi.ac.uk/˜lenov/meltinghome.html (Tm calculators)
http://frodo.wi.mit.edu/cgi-bin/primer/primer3_www.cgi
http://bibiserv.techfak.uni-bielefeld.de/genefisher2
http://www.premierbiosoft.com/qper/index

Typically, Q PCR involves the preparation of a standard curve for each amplified target nucleic acid sequence. Preparing a standard curve can indeed provide a good idea of the performance of the qPCR and thus serves as a quality control. The standard curve should cover the complete range of expected expression. Using standard material the standard curve should include at least 5 points of dilution, each of them in duplicate (at least). The 10-fold or 2-fold dilution range should cover the largest range of expression levels. Plotting these points on a standard curve, will determine the linearity, the efficiency, the sensitivity and the reproducibility of the assay. According to the present invention the standard curve is prepared from a genomic DNA sample. As used herein, “genomic DNA sample” or “gDNA” refers to a genomic DNA sample prepared from a DNA preparation. Methods for DNA purification are well known in the art. The genomic DNA may be prepared from a cell that is of the same organism than the cell that is used for preparing the nucleic acid sample of the invention (i.e. a human cell). Furthermore the cell from which the genomic sample is prepared must present the same ploidy than the cell used for preparing the nucleic acid sample of the invention; i.e. the cells present the same chromosomal abnormalities (e.g. in case of cancer cells). Typically, the genomic DNA sample is prepared from a cell for which the DII as defined above is about 1.

### Therapeutic Applications

The method of the present invention allows discriminating patients of having a good prognosis from patients having a poor prognosis. The methods of the present invention thus can be suitable for determining whether a patient is eligible or not to an anti-cancer treatment. An anti-cancer treatment typically consists of radiotherapy, chemotherapy, immunotherapy or a combination thereof. The treatment can also consist of an adjuvant therapy (i.e. treatment after chirurgical resection of the primary tumor) of a neoadjuvant therapy (i.e. treatment before chirurgical resection of the primary tumor).

In some embodiments, the methods of the present invention are suitable for determining whether a patient is eligible or not to a treatment with a chemotherapeutic agent. For example, when it is concluded that the patient has a poor diagnosis then the physician can take the choice to administer the patient with a chemotherapeutic agent.

The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cho lophosphamide, estramustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalamycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; amino levulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the methods of the present invention are suitable for determining whether a patient is eligible or not to targeted therapy. For example, when it is concluded that the patient has a poor diagnosis then the physician can take the choice to administer the patient with a targeted therapy.

Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names.

In some embodiments, the targeted therapy consists of administering the patient with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo [3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the methods of the present invention are suitable for determining whether a patient is eligible or not to a treatment with an immunotherapeutic agent. For example, when it is concluded that the patient has a poor diagnosis then the physician can take the choice to administer the patient with an immunotherapeutic agent.

The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the patient with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-a), IFN-beta (IFN-beta) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). The use of IFN-alpha, alone or in combination with other immunotherapeutics or with chemotherapeutics, has shown efficacy in the treatment of various cancers including melanoma (including metastatic melanoma), renal cancer (including metastatic renal cancer), breast cancer, prostate cancer, and cervical cancer (including metastatic cervical cancer).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Interleukins, alone or in combination with other immunotherapeutics or with chemotherapeutics, have shown efficacy in the treatment of various cancers including renal cancer (including metastatic renal cancer), melanoma (including metastatic melanoma), ovarian cancer (including recurrent ovarian cancer), cervical cancer (including metastatic cervical cancer), breast cancer, colorectal cancer, lung cancer, brain cancer, and prostate cancer.

Interleukins have also shown good activity in combination with IFN-alpha in the treatment of various cancers (Negrier et al., Ann Oncol. 2002 13(9):1460-8; Touranietal, J. Clin. Oncol. 2003 21(21):398794).

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in patients undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Colony stimulating factors have shown efficacy in the treatment of cancer, including melanoma, colorectal cancer (including metastatic colorectal cancer), and lung cancer.

Non-cytokine adjuvants suitable for use in the combinations of the present invention include, but are not limited to, Levamisole, alum hydroxide (alum), Calmette-Guerin bacillus (ACG), incomplete Freund's Adjuvant (IFA), QS-21, DETOX, Keyhole limpet hemocyanin (KLH) and dinitrophenyl (DNP). Non-cytokine adjuvants in combination with other immuno- and/or chemotherapeutics have demonstrated efficacy against various cancers including, for example, colon cancer and colorectal cancer (Levimasole); melanoma (BCG and QS-21); renal cancer and bladder cancer (BCG).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Monoclonal antibodies are used in the treatment of a wide range of cancers including breast cancer (including advanced metastatic breast cancer), colorectal cancer (including advanced and/or metastatic colorectal cancer), ovarian cancer, lung cancer, prostate cancer, cervical cancer, melanoma and brain tumours. Other examples include anti-CTLA4 antibodies (e.g. Ipilimumab), anti-PD1 antibodies, anti-PDLL antibodies, anti-TIMP3 antibodies, anti-LAG3 antibodies, anti-B7H3 antibodies, anti-B7H4 antibodies or anti-B7H6 antibodies.

Active specific immunotherapy typically involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, and lung cancer. Non-specific immunotherapeutics are useful in combination with cancer vaccines in order to enhance the body's immune response.

The immunotherapeutic treatment may consist of an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the patient's circulating lymphocytes, or tumor infiltrated lymphocytes, are isolated in vitro, activated by lymphokines such as IL-2 or transuded with genes for tumor necrosis, and readministered (Rosenberg et al., 1988; 1989). The activated lymphocytes are most preferably be the patient's own cells that were earlier isolated from a blood or tumor sample and activated (or “expanded”) in vitro. This form of immunotherapy has produced several cases of regression of melanoma and renal carcinoma.

In some embodiments, the methods of the present invention are suitable for determining whether a patient is eligible or not to a treatment with an radiotherapeutic agent. For example, when it is concluded that the patient has a poor diagnosis then the physician can take the choice to administer the patient with a radiotherapeutic agent.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

The invention will be further illustrated by the following figures and examples. However, these examples and figures should not be interpreted in any way as limiting the scope of the present invention.

### FIGURES

FIG. 1. Kaplan Meier survival curve and log-rank test according to the mutant status determined by ccfDNA analysis (n=97).

FIG. 2: A. Kaplan Meier survival curve and log-rank test according to Ref A KRAS determined by ccfDNA analysis (median 26 ng/mL of plasma, n=97).B. Kaplan Meier survival curve and log-rank test according to Ref A BRAF determined by ccfDNA analysis (median 27.6 ng/mL of plasma, n=97).

FIG. 3. Kaplan Meier survival curve and log-rank test according to DII BRAF determined by ccfDNA analysis (n=97).

FIG. 4: A. Kaplan Meier survival curve and log-rank test according to mA determined by ccfDNA analysis dichotomized around the median (3.2 ng/mL of plasma, n=43).B. Kaplan Meier survival curve and log-rank test according to mA determined by ccfDNA analysis dichotomized around 75% Q mA (22.9 ng/mL of plasma, n=43).

FIG. 5: A. Kaplan Meier survival curve and log-rank test according to mA % determined by ccfDNA analysis (n=43). The median mA % is 10.3% (0.51% to 64.2%). B. Kaplan Meier survival curve and log-rank test according to mA % dichotomized to the third quartile determined by ccfDNA analysis (n=43).

FIG. 6: A. Kaplan Meier survival curve and log-rank test according to CEA dichotomized around the median of 16.2 μg/L (n=97). B. Kaplan Meier survival curve and log-rank test according to CEA dichotomized around the standard threshold of 5 μg/L (n=97).

FIG. 7. Overall survival analysis on the entire cohort. A. Kaplan Meier survival curve and log-rank test according to CEA dichotomized around the standard threshold of 5 μg/L (n=83). B. Kaplan Meier survival curve and log-rank test according to the mutant status determined by ccfDNA analysis (n=97). C. Kaplan Meier survival curve and log-rank test according to Ref A KRAS determined by ccfDNA analysis dichotomized around the median (26 ng/mL of plasma, n=97. D. Kaplan Meier survival curve and log-rank test according to Ref A BRAF determined by ccfDNA analysis dichotomized around the median (27.6 ng/mL of plasma, n=97). Abbreviations: CEA, carcinoembryonic antigen; WT, wild-type for the KRAS and BRAF mutations tested; Ref A KRAS, total ccfDNA concentration as determined by targeting a WT KRAS sequence; Ref A BRAF, total ccfDNA concentration as determined by targeting a BRAF WT sequence.

FIG. 8. Overall survival analysis on the KRAS or BRAF mutant cohort. A. Kaplan Meier survival curve and log-rank test according to mA determined by ccfDNA analysis dichotomized around the median (3.06 ng/mL of plasma, n=43). B. Kaplan Meier survival curve and log-rank test according to mA % dichotomized to the 1^{st} tertile (4.14%) determined by ccfDNA analysis (n=43). C. Kaplan Meier survival curve and log-rank test according to Ref A KRAS dichotomized around the second tertile (Ref A KRAS=106.99 ng/mL, n=43. D. Kaplan Meier survival curve and log-rank test according to DII KRAS determined by ccfDNA analysis dichotomized around the 2^{nd} tertile (DII=0.20, n=43). E. Kaplan Meier survival curve and log-rank test according to CEA dichotomized around the standard threshold of 5 μg/L (n=36). Abbreviations: mA, mutant ccfDNA concentration; mA %, mutation load (% of mutant ccfDNA among total ccfDNA); RefA KRAS, total ccfDNA concentration as determined by targeting a WT KRAS sequence; DII KRAS, DNA integrity index determined using KRAS primer set; CEA, carcinoembryonic antigen.

### EXAMPLE 1

Material & Methods

Patients

106 metastatic colorectal cancer (mCRC) patients were analyzed from 3 clinical centers to investigate the predictive and prognostic value of qualitative and quantitative parameters determined from ccfDNA analysis. Eligible patients were male or female, age≧18 years, with histologically confirmed mCRC. Patients had measurable disease as defined by the Response Evaluation Criteria in Solid Tumors version 1.1 (RECIST v1.1) and were not treated by chemotherapy or radiotherapy in the month prior to the enrollment. Written, informed consent was obtained from all participants prior to the onset of the study. According to the Code de Sante Publique Article L1131-1 and next, no specific ethical approval is required for this type of study.

Specimen Characteristics and Preparation

Samples were handled accordingly with a pre-analytical guideline previously established by our group (24). 4 mL blood samples were collected in K3 EDTA tubes. Plasma was isolated within 1 hour following the blood drawing. The isolation process consisted in a 2 step centrifugation. First, blood tubes were centrifuged for 10 min in a Heraeus Multifuge LR centrifuge with a speed spin of 1200 g and a temperature of 4° C. Supernatant was collected, and buffy coat was avoided with precaution. The collected supernatant was centrifuged a second time for removing any possible remaining cells. This second centrifugation step was performed for 10 min, at 4° C. and with a speed spin of 16000 g. Plasma supernatant was then transferred in a 1.5 mL tube, extracted immediately after or stored at −20° C.

ccfDNA extraction was realized with the QIAGEN blood mini kit, and by following the “Blood and body fluid protocol”. During this extraction, 1 mL of plasma was processed sequentially in one column. Then, ccfDNA was eluted in 130 uL of elution buffer. Eluted ccfDNA was stored at −20° C. before Q-PCR analysis. Freeze-thawing was avoided to reduce fragmentation of eluted ccfDNA, and no extracts were conserved more than 3 months at −20° C.

Assay Methods

Intplex was a Q-PCR derived methodology developed for the analysis of ccfDNA. Detailed protocol and particularities of Intplex PCR method were detailed in previous works (23). Intplex was based on a nested diagram, where two short amplicons (60-100 bp±10 bp) were implemented among a larger amplicon (300±bp). One of the short amplicon was targeting a specific locus hotspot of interest (KRAS codon 12, 13 or BRAF codon 600 in our experiments, but it was applicable to other point mutations). The other short amplicon was designed for amplifying a WT sequence. This amplicon quantification gave an estimation of the total concentration in ccfDNA fragments (Ref A KRAS and Ref A BRAF). Primer design and validation were previously described (22).

Our Q-PCR thermal cycling protocol consisted of a polymerase activation step, and three repeated steps: a 3-min Hot-start Polymerase activation denaturation step at 95° C., followed by 40 repeated cycles at 95° C. for 10 s, and then at 60° C. for 30 s. Melting curves were obtained by increasing the temperature from 55° C. to 90° C. with a plate reading every 0.2° C. The concentration was calculated from Cq detected by Q-PCR and also a control standard curve on DNA of known concentration and copy number (Sigma-Aldrich). Serial dilutions of genomic DNA from human placenta cells (Sigma) were used as a standard for quantification and their concentration and quality was assessed using a Qubit spectrofluorimeter (Invitrogen). Q-PCR amplifications were carried out on a CFX96 instrument (Bio-Rad) using the CFX manager software (Bio-Rad). Intplex run were analysed with the CFX Manager Software (Bio-Rad). The positivity for a mutation, the concentration of mutant fragments (mA) and the mutated allele frequency (mA %) were determined with an analysis flowchart detailed in a precedent work of our team (21,22).

PCR run were assayed at least in duplicate in a 25 μL reaction volume. This master mix was constituted with 12.5 μL of master mix (Supermix SYBR green, Bio-Rad), 2.5 μL of each primer (0.3 pmol/mL, final concentration), 2.5 μL of PCR analysed water and 5 μL of template DNA. Non template controls were performed in each experiment for the different primer sets. Positive controls for mutation assessment were also added in each PCR run. These controls are genomic DNA from cell-line with known mutation. The respective correspondence between cell lines and the corresponding mutation was further detailed: HCT-116 for the G13D KRAS mutation, SW620 for the G12V KRAS mutation, A549 for the G12S KRAS mutation, LS174T for the G12D KRAS mutation, MiaPaca2 for the G12C mutation, SW1116 for the G12A KRAS mutation, and HT29 for the V600E BRAF mutation. Synthetic DNA bearing the KRAS sequence of interest (Horizon Discovery Ltd.) was used as a positive control for KRAS G12R. Evaluation of the sensitivity level of our method was conducted on genomic DNA. From each targeted mutation, a corresponding positive control was added and its sensitivity was evaluated. DNA from the cells harboring targeted mutation was serially diluted six times into high-concentrated WT genomic DNA from human placenta (Sigma Aldrich) up to a dilution of 0.2 mutated copies in 20,000 WT copies.

The degree of ccfDNA fragmentation was assessed simultaneously on targeted KRAS and BRAF hotspots from each plasma samples by calculating the DNA Integrity Index (DII KRAS and DII BRAF). The DII was determined by calculating the ratio of the concentration determined by using the primer set amplifying a large target (300 AO bp) to the concentration determined by using the primer set amplifying a short target (<100 bp).

Study Design and Statistics

Blood collection for ccfDNA analysis was performed near to the date of first metastatic diagnosis (median: 1.3 month of delay after first metastatic diagnosis). Carcino Embryonic Antigen (CEA) measure was performed in the two months preceding or following the blood sampling for ccfDNA analysis. Data were summarized by frequency for categorical variables and by median and range values for continuous variables. Overall Survival (OS) was calculated from the date of first metastatic diagnosis to the date of death and Progression Free Survival (PFS) was calculated from the date of first metastatic diagnosis to the date of progression. Survival rates were estimated using the Kaplan-Meier method. In univariate analysis, the log-rank test was used to identify prognostic variables. Univariate analysis was performed for each ccfDNA parameter (Ref A KRAS, Ref A BRAF, mA, mA %, DII KRAS, DII BRAF), for CEA and clinical parameters. Significant parameters for OS in univariate analysis (P<0.1): BRAF mutant, Ref A KRAS and CEA were included in a multivariate Cox proportional hazards model. Statistical analysis was performed using the STATA 11.0 software (StataCorp LP, College Station, Tex., USA).

Results

Patient's Characteristics

Patient's baseline characteristics, number and localization of metastasis, number of previous lines of therapy are listed in Table 1.

TABLE 1
Patient's baseline characteristics.
Patient Characteristics (N = 97)

| Characteristics | No. | % |
|---|---|---|
| Centre | | |
| CRLC Montpellier | 25 | 25.8 |
| CHU Clermont-Ferrand | 22 | 22.7 |
| CHU Limoges | 50 | 51.5 |
| Gender | | |
| Male | 58 | 59.8 |
| Female | 39 | 40.2 |
| Age, years | | |
| Median (Range) | 66.6 | 36-87 |
| Missing | 4 | |
| Primary tumor site | | |
| Right colon | 22 | 22.7 |
| Left colon | 41 | 42.2 |
| Rectum | 34 | 35.1 |
| Chemotherapy | | |
| Naive | 62 | 63.9 |
| Neo-adjuvant/Adjuvant | 22 | 22.7 |
| Palliative (n = 13) | | |
| 1 line, metastatic | 4 | 4.1 |
| ≧2 lines, metastatic | 9 | 9.3 |
| Primary tumor site in place | 53 | 54.6 |
| No. of metastatic sites in place | | |
| 1 | 51 | 54.3 |
| >1 | 43 | 45.7 |
| Missing | 3 | |

106 mCRC patients were included in this study, during the period comprised between July 2010 and December 2012. 8 patients were excluded of the study because of irrespective inclusion criteria and 1 was lost of sight. The median follow-up time was 36 months (1 day to 104 months). Median OS was 22 months which is consistent with current data on overall survival of mCRC patients (from 18 to 24 months). 1 of the 106 mCRC patients was lost of sight and 8 were not included because of non inclusion criteria, and so were not evaluable for overall survival in this study. Ref A KRAS, Ref A BRAF, DII KRAS and DII BRAF were available for 97mCRC patients. CEA values were determined in 83 mCRC patients. 43 mutations on KRAS or BRAF have been identified, and mA and mA % were determined in all of these 43 KRAS or BRAF mutant mCRC patients.

ccfDNA Analysis and CEA Values:

The median concentrations Ref A KRAS and Ref A BRAF were respectively 26 ng/mL [2.58-1386.9] and 27.6 ng/mL [1.12-1227.2] of plasma. The median determined DII ratio for KRAS and BRAF were both 0.1. ccfDNA analysis revealed that 38 mCRC patients (37% of the cohort) were mutant for one the 7 tested KRAS mutations and 5% of the cohort exhibited a BRAFV600E mutation. Those results were fully validated in a blinded study comparing with the mutant status determined from tumor tissue (22). In those patients, the median mA concentration detected was 3.2 ng/mL [0.04-507] of plasma. mA % median mutation load was 10.5% [0.51-64.2]. Median CEA concentration was determined from 83 mCRC patients at 16.2 μg/L [0.57-19997] of plasma. Detailed data for each patient are presented in Table 2.

TABLE 2
Median values of studied parameters

| | |
|---|---|
| KRAS | BRAF |

| | | |
|---|---|---|
| Median ccfDNA concentration (refA in ng/mL of | 26 | 27.6 |
| plasma) | | |
| Mutation frequency in cohort (in %) | 37 | 5 |
| Median DII | 0.1 | 0.1 |

| | |
|---|---|
| Median mutant ctDNA concentration (ng/mL of | 3.2 |
| plasma) | |
| Median mA % | 10.5 |
| Median CEA concentration (μg/L of plasma) | 16.2 |

Relation Between the Mutational Profile and Overall Survival

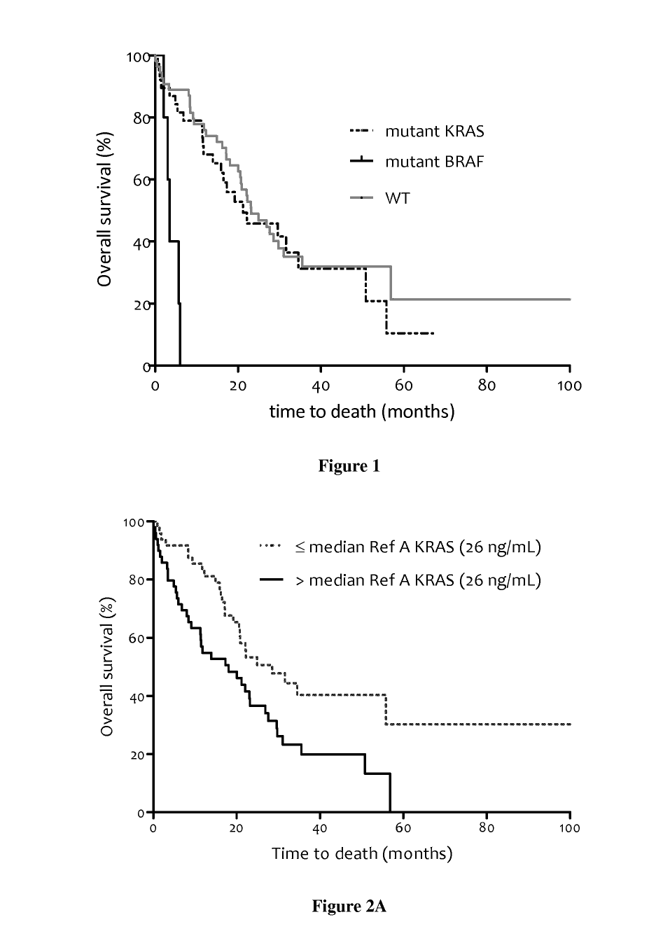

Patients WT for KRAS and BRAF had a median overall survival of 21.9 months compared to 20.9 months for mutant KRAS mCRC patients and 3.4 months for BRAF mutant mCRC patients. For each mutant status, Kaplan-Meier survival curves were calculated (FIG. 1). Surprisingly, there was no significant differences in the OS between WT (n=54) and KRAS mutant mCRC patients (p=0.675, RR=1.11). However, there was a tendency to a significant difference in the OS between patients with a BRAF mutation and patients with a KRAS mutation. WT patients exhibited also a tendency to have a significant different OS with BRAF mutated patients (p<0.0001, RR=8.93).

Higher Total ccfDNA Concentration (refA KRAS and BRAF) is Correlated to a Decrease in Overall Survival.

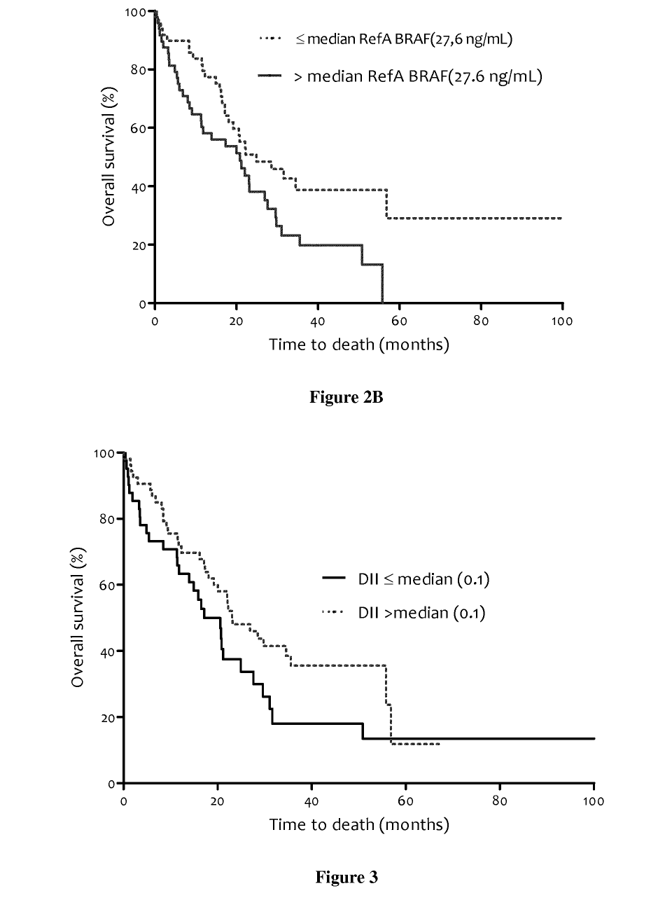

Patients with Ref A KRAS below the median of 26 ng/mL of plasma had a median overall survival of 24.5 months while it was 17 months for patients with Ref A KRAS higher than the median; p=0.012, RR=1.88 (FIG. 2A). We observed also a significant difference when analyzing Ref A BRAF: patients with Ref A BRAF below 27.6 ng/mL of plasma had a median overall survival of 24.5 months while it was 20.5 months for mCRC patients with higher levels; p=0.025, RR=1.76 (FIG. 2B).

ccfDNA Fragmentation (DII KRAS and DII BRAF) and Overall Survival.

When studying DII ratio, we have determined that mCRC patients with a higher DII than the median value (0.1) had a higher median overall survival than patients with lower level. mCRC patients with a DII BRAF greater than 0.1 had a median overall survival of 23.2 months while it was 17.2 months for patients with higher fragmentation; p=0.1, RR=1.5 (FIG. 3). This trend was also observed when studying DII KRAS, median overall survival for mCRC patients with DII higher than 0.1 was 23 months and decreased to 17.3 months for mCRC patients with DII below 0.1. It seemed that a higher level of fragmentation had a tendency to be correlated with a worse prognosis.

Higher ccfDNA Concentrations (mA) are Correlated with Shorter Overall Survival.

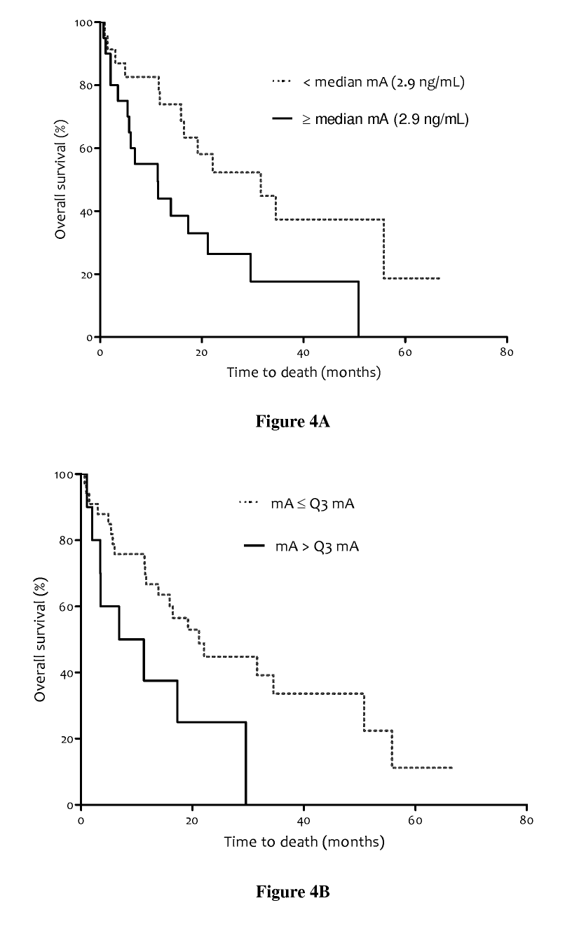

KRAS or BRAF mutant mCRC patients with a mA below 3.2 ng/mL of plasma (median cohort concentration of mA in ng/mL of plasma) had a median overall survival of 31.1 months (FIG. 4A). For mutant mCRC patients with higher levels, the median overall survival was 11.1 months; p=0.015, RR=2.54. When studying only the third quartile, this observation was confirmed (p=0.025, RR=2.5) (FIG. 4B). The presence of BRAF-V600E mutation is known to be strongly correlated with a decrease in patient's survival. In order to avoid this influence of BRAF V600E mutation bad prognosis on overall survival analysis, we have also analyzed this parameter exclusively in KRAS mutant mCRC patients (n=38). We have found that mA was still correlated with outcome: mCRC patients with higher mA than the median presented a median overall survival of 31.6 and patients with lower mA had an overall survival median of 13.9 months, p=0.05, RR=2.27.

Patients with High Mutation Load have Reduced Overall Survival.

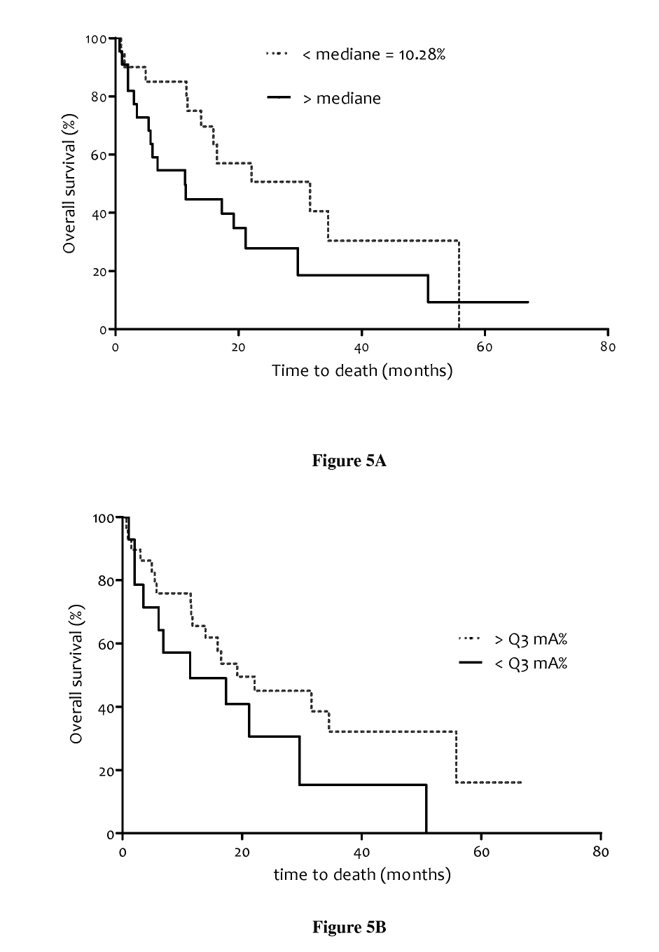

Mutant mCRC patients with a mA % lower than 10.3% had a median overall survival of 31.1 months while it was 11.4 months for mCRC patients with higher levels, p=0.14, RR=2.7 (FIG. 5A). Even if there were no significant differences between the two groups, this tendancy was confirmed with different thresholds: when studying the first quartile (2.4), median overall survival of mCRC patients with low mA % was 31.6 months and 16.5 months for patients with higher level. When analyzing the third quartile (17.9), patients with low mA % presented a median overall survival of 19.2 months and patients with higher level a median of 11.3 months. (FIG. 5B) A higher cohort of mutated patients would help to conclude on the significativity or not of this parameter for the overall survival analysis. After removing the five mCRC patients exhibiting a BRAF V600E mutation, we observed that there was a trend for patients with low mA % presenting a median overall survival of 31.6 months compared to the median overall survival of patients with higher levels which decreased to 19.2 months. p=0.32, RR=1.64.

Relation Between CEA and Overall Survival.

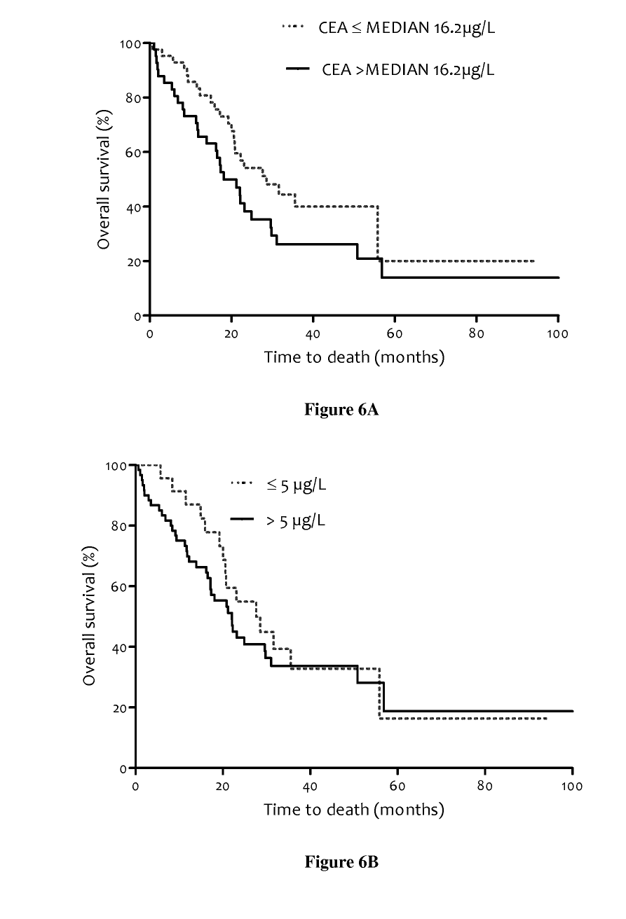

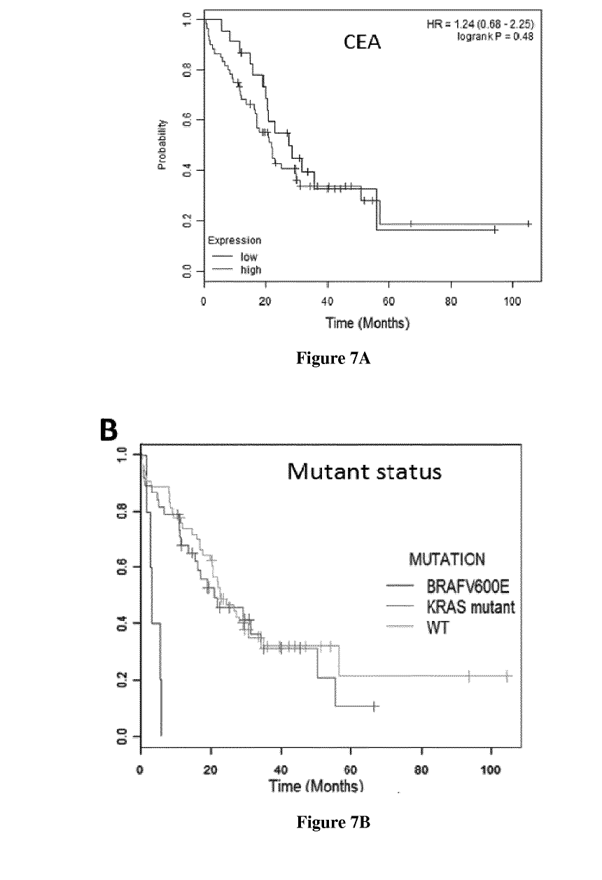

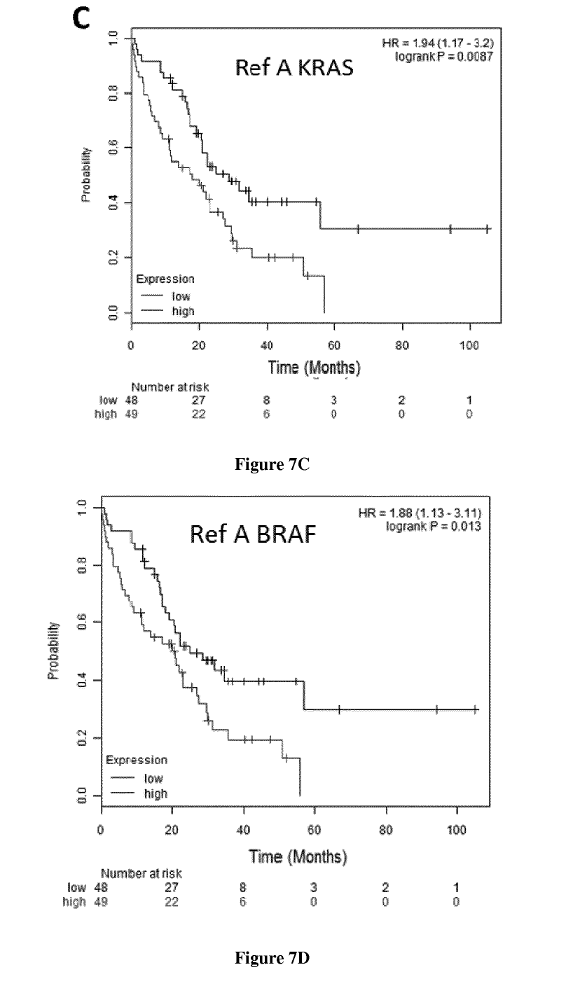

Patients with CEA level higher than the median concentration (16.2 μg/L) presented a median overall survival of 28.1 months and it was 17.8 months for patients with lower levels, p=0.088, RR=1.60. Nevertheless, patients with higher CEA level than the current clinical threshold of significance (5 μg/L), had a median overall survival of 27.2 months compared to patients with lower levels presenting a median overall survival of 21.7 months; p=0.48, RR=1.24. Kaplan-Meier curves are shown in FIGS. 6A and 6B.

Multivariate Analysis

ccfDNA parameters highly significant in univariate analysis: BRAF mutant, Ref A KRAS and current routine standard in clinical practice, CEA, were included in a multivariate Cox proportional hazards model on the entire cohort. Results show that the total cfDNA concentration appeared statistically a strong independent prognostic factor (P=0.034, RR=1.73) as well as BRAF mutant status (p=0.002, RR=7.33).

Progression Free Survival Analysis

On a cohort of 72 mCRC patients, we showed the relation between different ccfDNA parameters and PFS. We showed that Ref A, mA and mA % were significantly associated with outcome. Nevertheless, fragmentation does not seem to correlate with progression. Data are summarized in Table 3.

TABLE 3

| | RR | p-value | significativity |
|---|---|---|---|
| BRAF mutant | 7.5 | 0.0058 | ** |
| (n = 5) vs KRAS | | | |
| mutant (n = 27) | | | |
| or KRAS/BRAF | | | |
| WT (n = 40) | | | |
| Ref A KRAS | 1.8 | 0.09 | * |
| Ref A BRAF | 1.8 | 0.04 | * |
| DII KRAS | 1.1 | 0.57 | ns |
| DII BRAF | 1.0 | 0.93 | ns |
| mA | 2.4 | 0.08 | * |
| (dichotomisation | | | |
| to the median) | | | |
| mA | 3.8 | 0.02 | ** |
| (dichotomization | | | |
| to the third | | | |
| quartile) | | | |
| mA % | 2.8 | 0.02 | ** |
| CEA | 0.85 | 0.72 | ns |

### EXAMPLE 2

In EXAMPLE 1, we examined overall survival (OS) of 106 mCRC patients from three clinical centers; this was the largest cohort of mCRC patients studied for potential prognostic interest of ccfDNA analysis. Total ccfDNA concentration, determination of the main KRAS and BRAF mutations, mutant ccfDNA concentration, the proportion of mutation, and ccfDNA integrity were simultaneously determined for the first time in all patients in relation to prognosis. We investigated the value of these parameters according to OS by univariate and multivariate analysis. The results were compared to the prognostic value of the CEA. In order to reinforce the prognostic value of ccfDNA analysis and the necessity to study different parameters of ccfDNA: concentration, fragmentation, mutation detection and mutation quantification, we added acute univariate and multivariate analysis of OS in the entire cohort of patients and acute univariate and multivariate analysis of OS in mutant subgroup of patients. The results were still compared to prognostic value of CEA, the current biomarker used in clinical practice. We decided to investigate this prognostic value following current clinical standards in order to translate easily the analysis at patient's bedside.

Material and Methods:

We added univariate analysis of OS using different thresholds for each ccfDNA parameter: first tertile, median and second tertile since acute statistical analysis revealed that those thresholds were optimal.

We added analysis of OS in the subgroup of KRAS/BRAF mutant patients (n=43). In each group of patients, mutant ccfDNA concentration, of mutation, and ccfDNA integrity were simultaneously analyzed for their relation with OS and compared to the prognostic value of CEA. This subgroup analysis was realized following univariate analysis statistical method in the cohort of KRAS/BRAF mutant patients (n=38 KRAS mutant patients and n=5 BRAF mutant patients). This subgroup analysis was realized following multivariate analysis statistical method in the cohort of KRAS/BRAF mutant patients (n=38 KRAS mutant patients and n=5 BRAF mutant patients).

Entire analysis of the prognostic value of ccfDNA analysis in patients suffering from cancer was realized following the official guideline for prognostic studies of biomarkers: REMARK (recommendations for tumor MARKer prognostic studies).

Results:

OS Analysis in the Entire Cohort

Univariate analysis in the entire cohort is depicted in Table 4.

Relation Between CEA and Overall Survival:

Patients with lower CEA levels than the current clinical threshold of significance (5 μg/L) had a median OS of 27.2 months while patients with higher levels had a median OS of 21.7 months (p=0.48, RR=1.24) (FIG. 1A and Table 4). Such difference is not significative.

Correlation of Mutant Status with Overall Survival:

Patients WT for KRAS exon 2 codon 12/13 and BRAFV600E showed a median OS of 21.9 months compared to 20.9 months for KRAS-mutant patients (n=38) and 3.4 months for BRAF-mutant mCRC patients (n=5) (Table 4 and FIG. 1B). There was a statistically high significant difference between the median OS of BRAF-mutant patients (n=5) and KRAS-mutant patients (n=38) (p<0.0001, RR=6.106). Median OS of WT patients showed a statistically high significant difference when compared to BRAF-mutant patients (p<0.0001, RR=8.93).

Higher Total ccfDNA Concentration is Statistically Correlated with a Decrease in Overall Survival:

Patients with Ref A KRAS (total ccfDNA concentration determined with KRAS primer set) below the median of 26 ng/mL of plasma had a median OS of 28.5 months while it was 18.07 months for patients with Ref A KRAS higher than the median (p=0.0087, RR=1.94) (FIG. 1C). This was confirmed when studying Ref A BRAF (total ccfDNA concentrations determined with BRAF primers sets): patients with Ref A BRAF below 27.6 ng/mL of plasma had a median OS of 24.5 months compared to 20.5 months for mCRC patients with higher levels (p=0.013, RR=1.55) (FIG. 1D). Statistically significant differences were also determined when comparing groups to the 2^{nd} tertile value of Ref A KRAS or BRAF (p=0.013 and 0.011, respectively) (Table 4).

CcfDNA Fragmentation and Overall Survival:

mCRC patients showing higher DII KRAS (DNA integrity index determined with KRAS primer set) than the median value (0.12) had a higher median OS than patients with lower levels (23.07 months vs. 17.3 months) (Table 4). This observation was the same when analyzing DII BRAF (DNA integrity index determined with BRAS primer set): mCRC patients with a DII higher than the median (0.11) had a median OS of 23.07 months compared to 17.17 months for mCRC patients with highly fragmented DNA. When analyzing the first tertile of DII BRAF (0.07), a significant difference was shown, although not statistically, between the two groups of patients (p=0.12) (Table 4). It seemed that a higher level of fragmentation had a tendency to be correlated with worse prognosis.

TABLE 4
Overall survival univariate analysis on the entire cohort and the subgroup
of mutant cohort for mA and mA %. Each parameter generated by ccfDNA
analysis is tested by dichotomization of the cohort on the 1^{st} tertile, the
median and the 2^{nd} tertile. CEA is dichotomized around the standard
threshold of 5 μg/L.

| | | | | |
|---|---|---|---|---|
| | Median | | | |
| Death | OS | | | |
| occurrence | (Mo) | RR | CI 95% | p-value |

KRASmutant status

| | | | | | |
|---|---|---|---|---|---|
| WT | 40/60 | 21.9 | 1 | [0.67-1.85] | 0.675 |
| mutant | 24/38 | 20.9 | 1.11 | | |

BRAF mutant status

| | | | | | |
|---|---|---|---|---|---|
| WT | 59/92 | 21.9 | 1 | | |
| mutant | 5/5 | 3.4 | 8.93 | [3.13-25.4] | <0.0001 |

KRAS or BRAF mutant

| | | | | | |
|---|---|---|---|---|---|
| KRAS mutant | 24/38 | 20.9 | 1 | [5.72-6.487] | <0.0001 |
| BRAF mutant | 5/5 | 3.4 | 6.106 | | |

Ref A KRAS (ng/ml)

| | | | | | |
|---|---|---|---|---|---|
| ≦1st tertile: 15.6 | 19/32 | 22.23 | 1 | [0.465-1.635] | 0.253 |
| >1st tertile: 15.6 | 45/65 | 21.17 | 1.05 | | |
| ≦median: 26.0 | 26/49 | 28.5 | 1 | [1.17-3.20] | 0.0087 |
| >median: 26.0 | 38/48 | 18.07 | 1.94 | | |
| ≦2nd tertile: 47.5 | 39/64 | 23.17 | 1 | [1.14-3.15] | 0.013 |
| >2nd tertile: 47.5 | 25/33 | 13.9 | 1.89 | | |

Ref A BRAF (ng/ml)

| | | | | | |
|---|---|---|---|---|---|
| ≦1st tertile: 13.6 | 19/32 | 22.23 | 1 | | |
| >1st tertile: 13.6 | 45/65 | 21.17 | 1.05 | [0.465-1.635] | 0.2 |
| ≦median: 27.6 | 28/49 | 24.5 | 1 | [1.13-3.11] | 0.013 |
| >median: 27.6 | 36/48 | 20.5 | 1.88 | | |
| ≦2nd tertile: 48 | 39/64 | 24.9 | 1 | | |
| >2nd tertile: 48 | 25/33 | 13.9 | 1.8 | [1.15-3.19] | 0.011 |

mA

| | | | | | |
|---|---|---|---|---|---|
| ≦1st tertile: 1.06 | 10/14 | 22.1 | 1 | [1.125-2.055] | 0.57 |
| >1st tertile: 1.06 | 19/29 | 13.9 | 1.59 | | |
| ≦median: 3.06 | 13/22 | 31.6 | 1 | [1.25-5.93] | 0.0089 |
| >median: 3.06 | 16/21 | 11.3 | 2.72 | | |
| ≦2nd tertile: 7.53 | 16/28 | 22.1 | 1 | [1.28-5.78] | 0.0071 |
| >2nd tertile: 7.53 | 13/15 | 6.83 | 2.72 | | |

mA (%)

| | | | | | |
|---|---|---|---|---|---|
| ≦1st tertile: 4.14 | 7/14 | 34.53 | 1 | [0.97-5.44] | 0.053 |
| >1st tertile: 4.14 | 22/29 | 13.9 | 2.29 | | |
| ≦median: 10.72 | 13/22 | 31.6 | 1 | [0.82-3.621] | 0.15 |
| >median: 10.72 | 16/21 | 11.4 | 1.72 | | |
| ≦2nd tertile: 15.9 | 17/28 | 22.1 | 1 | | |
| >2nd tertile: 15.9 | 12/15 | 11.3 | 1.93 | [0.9-4.12] | 0.08 |

DII KRAS

| | | | | | |
|---|---|---|---|---|---|
| ≦1st tertile: 0.07 | 20/31 | 20.8 | 1 | [0.64-1.90] | 0.72 |
| >1st tertile: 0.07 | 44/65 | 22.1 | 1.1 | | |
| ≦median: 0.12 | 32/49 | 17.3 | 1 | [0.72-1.93] | 0.51 |
| >median: 0.12 | 32/47 | 23.07 | 1.17 | | |
| ≦2nd fertile: 0.23 | 44/64 | 21.17 | 1 | [0.77-2.13] | 0.33 |
| >2nd tertile: 0.23 | 20/32 | 23.07 | 1.28 | | |

DII BRAF

| | | | | | |
|---|---|---|---|---|---|
| ≦1st tertile: 0.07 | 25/34 | 20.6 | 1 | [0.89-2.6] | 0.12 |
| >1st tertile: 0.07 | 39/60 | 22.23 | 1.53 | | |
| ≦median: 0.11 | 33/49 | 17.17 | 1 | [0.83-2.25] | 0.2 |
| >median: 0.11 | 31/47 | 23.07 | 1.37 | | |
| ≦2nd tertile: 0.20 | 41/62 | 20.8 | 1 | [0.62-1.7] | 0.9 |
| >2nd fertile: 0.20 | 23/32 | 22.23 | 1.03 | | |

CEA (μg/L)

| | | | | | |
|---|---|---|---|---|---|
| ≦5 | 15/23 | 27.2 | 1 | | |
| >5 | 38/61 | 21.7 | 1.24 | [0.68-2.25] | 0.48 |

OS: overall survival.
mo: months.
RR: relative risk.
CI: confidence interval

Multivariate Analysis in the Entire Cohort (No Difference with Initial Data)

CcfDNA parameters that were found to be highly significant in univariate analysis, BRAF mutant, Ref A KRAS (total ccfDNA concentration), CEA, and current routine standards in clinical practice were included in a multivariate Cox proportional hazards model on the entire cohort. Results showed that total ccfDNA concentration appeared statistically as a strong independent prognostic factor (p=0.034, RR=1.73), as well as BRAF-mutant status (p=0.002, RR=7.33).

Univariate Analysis in the Mutant Cohort

Higher Mutant ccfDNA Concentrations are Statistically Correlated with Shorter Overall Survival.

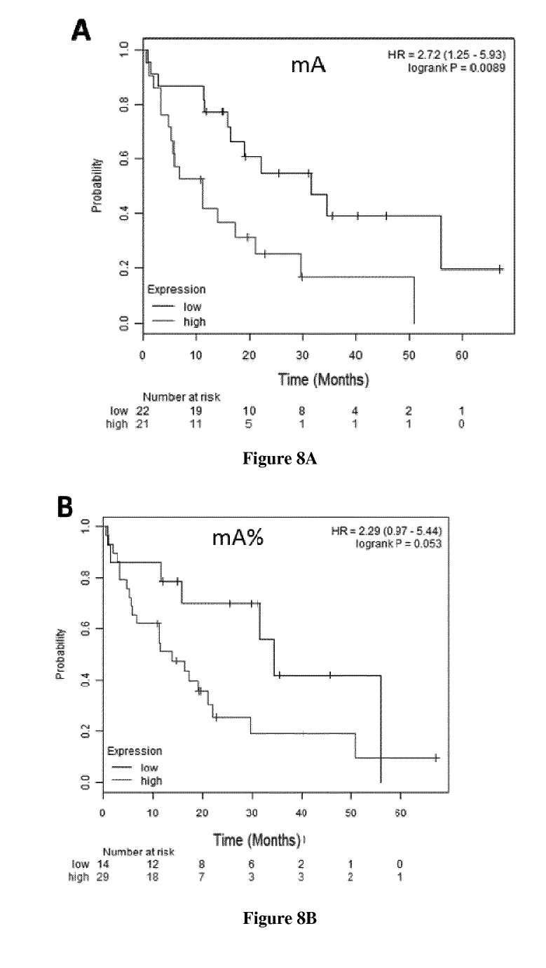

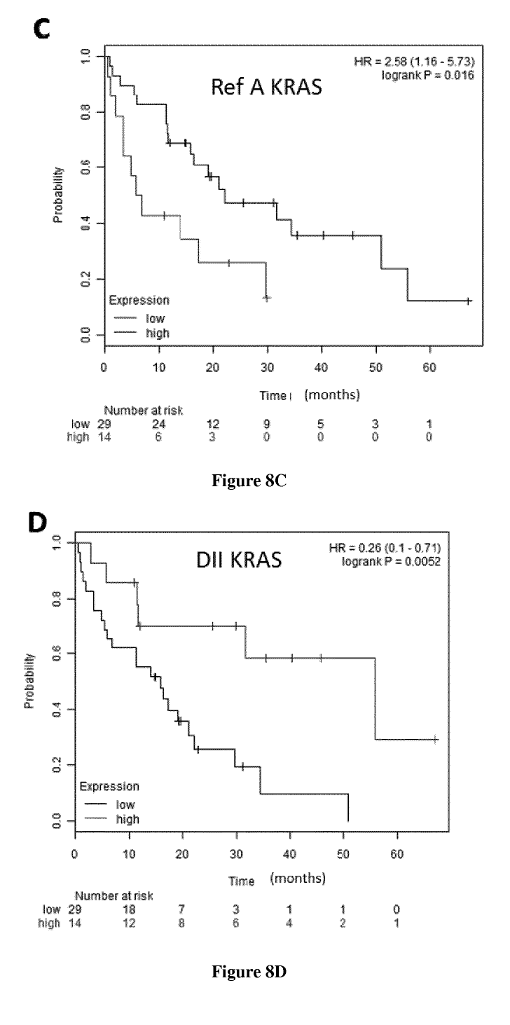

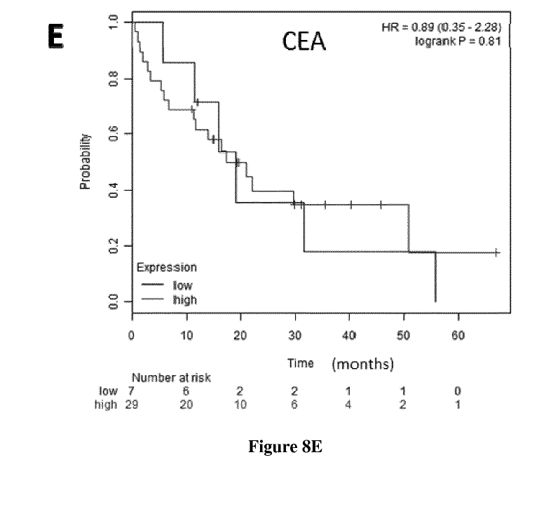

KRAS or BRAF mutant mCRC patients with a mA (mutant ccfDNA concentration) below 3.06 ng/mL of plasma (median cohort concentration of mA in ng/mL of plasma) had a median OS of 31.6 months (FIG. 8A) while the median OS was 11.3 months for patients with higher levels than the median mA (p=0.0089, RR=2.7). This observation was confirmed with the 2^{nd} tertile as threshold (p=0.0071, RR=2.7) (Table 4). In order to avoid the influence of BRAF V600E mutation poor prognosis on OS analysis, we analyzed this parameter exclusively in KRAS-mutant mCRC patients (n=38). mA was still correlated with outcome: mCRC patients with higher mA than the 2^{nd} tertile presented a median OS of 31.6 compared to a median OS of 11.4 months in patients with a lower mA (p=0.0067, RR=2.78) (data not shown).

Patients with High Mutation Load have Statistically Reduced OS.

Mutant mCRC patients with mutation loads (mA %) lower than the median value (10.72%) had a median OS of 31.6 months compared to 11.4 months for mCRC patients with higher levels (p=0.15, RR=2.8). Despite huge difference in OS between the two groups, there was no statistical difference. This tendancy was confirmed with different thresholds: when studying the first tertile (4.14%), the median OS of mCRC patients with low mA % was 34.6 months compared to 13.9 months for patients with higher levels (p=0.05, RR=2.29) (FIG. 8B). When analyzing the second tertile (15.9%), patients with low mA % presented a median OS of 22.1 months compared to a median OS of 11.3 months for patients with higher levels (p=0.08, RR=1.93)(Table 4). When the five mCRC patients exhibiting a BRAF V600E mutation were removed from the evaluated cohort, we observed that there was a trend showing a difference in OS for patients with low mA % with a median OS of 31.6 months compared to patients showing higher levels as the median OS decreased to 17.3 months (p=0.11, RR=1.827) (data not shown).

Higher Total ccfDNA Concentration and Fragmentation are Correlated with Decreased Os:

Ref A KRAS and DII KRAS (total ccfDNA concentration and DNA integrity index determined with KRAS primer sets) were highly significant in univariate analysis in the mutant cohort (p=0.016 and 0.005 respectively, n=43) (FIGS. 8C and 8D) while CEA was not significant (p=0.81) (FIG. 8E).

Multivariate Analysis in the Subgroup of Mutant Cohort

Multivariate Cox proportional hazards model revealed that Ref A KRAS appeared as an independent prognostic factor (p=0.057, RR=3.67) and that DII KRAS appeared as a strong independent prognostic factor (p=0.0072, RR=3.57). Note that when studying DII KRAS in the exclusive WT patients cohort, it did not appear of prognostic value (p=0.67, n=54, data not shown).

### REFERENCES

Throughout this application, various references describe the state of the art to which this invention pertains. The disclosures of these references are hereby incorporated by reference into the present disclosure.
1. World Cancer Research Fund International, 2012.
2. Poston G J et al, Urgent need for a new staging system in advanced colorectal cancer. J Clin Oncol. 2008 October 10; 26(29):4828-33.
3. Schwarzenbach et al, Cell-free nucleic acids as biomarkers in cancer patients. Nat Rev Cancer. 2011 June; 11(6):426-37.
4. Lecomte et al, Detection of free-circulating tumor-associated DNA in plasma of colorectal cancer patients and its association with prognosis. Int J Cancer. 2002 August 10; 100(5):542-8.
5. Wang J Y et al, Molecular detection of APC, K-ras, and p53 mutations in the serum of colorectal cancer patients as circulating biomarkers. World J Surg. 2004 July; 28(7):721-6.
6. Bidard F C et al, Detection rate and prognostic value of circulating tumor cells and circulating tumor DNA in metastatic uveal melanoma. Int J Cancer. 2014 March 1; 134(5):1207-13.
7. Bettegowda et al, Detection of circulating tumor DNA in early- and late-stage human malignancies. Sci Transl Med. 2014 February 19; 6(224).
8. Dawson S J et al, Circulating tumor DNA to monitor metastatic breast cancer. N Engl J Med. 2013 July 4; 369(1):93-4.
9. Sirera R et al, Circulating DNA is a useful prognostic factor in patients with advanced non-small cell lung cancer. J Thorac Oncol. 2011 February; 6(2):286-90.
10. Sunami E et al, Multimarker circulating DNA assay for assessing blood of prostate cancer patients. Clin Chem. 2009 March; 55(3):559-67.
11. Perkins et al, Multi-purpose utility of circulating plasma DNA testing in patients with advanced cancers. PLoS One. 2012; 7(11):e47020.
12. Philipp A B et al, Prognostic role of methylated free circulating DNA in colorectal cancer. Int J Cancer. 2012 November 15; 131(10):2308-19.
13. Lee H S et al, Circulating Methylated Septin 9 Nucleic Acid in the Plasma of Patients with Gastrointestinal Cancer in the Stomach and Colon. Transl Oncol. 2013 June 1; 6(3):290-6.
14. Jung K et al, Cell-free DNA in the blood as a solid tumor biomarker—a critical appraisal of the literature. Clin Chim Acta. 2010 November 11; 411(21-22):1611-24.
15. Fleischhacker M et al, Circulating nucleic acids (CNAs) and cancer—a survey. Biochim Biophys Acta. 2007 January; 1775(1):181-232.
16. Marzese D M et al, Diagnostic and prognostic value of circulating tumor-related DNA in cancer patients. Expert Rev Mol Diagn. 2013 November; 13(8):827-44.
17. Umetani N et al, Prediction of breast tumor progression by integrity of free circulating DNA in serum. J Clin Oncol. 2006 September 10; 24(26):4270-6.
18. Thierry A R et al, Origin and quantification of circulating DNA in mice with human colorectal cancer xenografts. Nucleic Acids Res. 2010 October; 38(18):6159-75.
19. Mouliere F et al, High fragmentation characterizes tumour-derived circulating DNA. PLoS One. 2011; 6(9):e23418.
20. Mouliere F et al, Circulating Cell-Free DNA from Colorectal Cancer Patients May Reveal High KRAS or BRAF Mutation Load. Transl Oncol. 2013 June 1; 6(3):319-28.
21. Mouliere F et al, Multi-marker analysis of circulating cell-free DNA toward personalized medicine for colorectal cancer. Mol Oncol. 2014 March 24
22. Thierry A R et al, Clinical validation of the detection of KRAS and BRAF mutations from circulating tumor DNA. Nat Med. 2014 April; 20(4):430-5.
23. Diehl et al, Circulating mutant DNA to assess tumor dynamics. Nat Med. 2008 September; 14(9):985-90.
24. El Messaoudi S et al, Circulating cell-free DNA: preanalytical considerations. Clin Chim Acta. 2013 September 23; 424:222-30.

1. A method for predicting the survival time of a patient suffering from a cancer comprising the steps of i) extracting the cell free nucleic acids from a sample obtained from the patient, ii) determining the level of the mutant nucleic acids liable to be present in the extracted cell free nucleic acids, iii) comparing the level determined at step ii) with a predetermined reference value and iv) concluding that the patient will a short survival time when the level determined at step ii) is higher than the predetermined reference value or concluding that the patient will have a long survival time when the level determined at step ii) is lower than the predetermined reference value.

2. The method of claim 1 wherein the mutation directly contributes to the initiation of the malignant transformation.

3. The method of claim 1 wherein the mutation is located in a gene selected from the group consisting of KRAS, BRAF, NRAS, TP53, APC, MSH6, NF1, PIK3CA, SMAD4, EGFR, CDKN2A, IDH1, PTEN, SMARCB1, CTNNB1, HNF1A, VHL, ATM, EZH2, RET, NRAS, PTCH1, KIT, NF2, PDGFRA, PPP2R1A, STK11, MLL3, FOXL2, GNAS, HRAS, FGFR3, PTCH1, and CDH1.

4. The method of claim 1 wherein the mutation is a KRAS mutation.

5. The method of claim 4 wherein the KRAS mutation is selected from the group consisting of G12C, G12D, G13D, G12R, and G12V.

6. The method of claim 1 wherein the mutation is a BRAF mutation.

7. The method of claim 6 wherein the BRAF mutation is V600E.

8. The method of claim 1 wherein the level of the mutant nucleic acids is determined by Q-PCR.

9. The method of claim 1 wherein the level of the mutant nucleic acids is determined by amplifying a target nucleic acid sequence having less than 100 base pairs and which comprises the mutation of interest.

10. The method of claim 9 wherein the target nucleic acid sequence for determining the level of the mutant nucleic acids has a length of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; or 110 base pairs.

11. The method of claim 1 which is performed for at least 2 mutations wherein for each mutation (M)n the level of the mutant nucleic acids (ELM)n is determined and compared with its corresponding predetermined reference value (ELRM)n and wherein the higher the number of (ELM)n are higher than their corresponding predetermined values (ELRM)n, the shorter will be the survival time of the patient.

12. A method for predicting the survival time of a patient suffering from a cancer comprising the steps of i) extracting the cell free nucleic acids from a sample obtained from the patient, ii) determining the level of the mutant nucleic acids liable to be present in the extracted cell free nucleic acids, iii) determining the total concentration of cell free nucleic acids, iv) calculating the ratio of the level determined at step ii) to the concentration determined at step iii), v) comparing ratio determined at step iv) with a predetermined reference value and vi) concluding that the patient will a short survival time when the ratio determined at step iv) is higher than the predetermined reference value or concluding that the patient will have a long survival time when the level determined at step iv) is lower than the predetermined reference value.

13. The method of claim 12 wherein the mutation of interest is located in a gene selected from the group consisting of KRAS, BRAF, NRAS, TP53, APC, MSH6, NF1, PIK3CA, SMAD4, EGFR, CDKN2A, IDH1, PTEN, SMARCB1, CTNNB1, HNF1A, VHL, ATM, EZH2, RET, NRAS, PTCH1, KIT, NF2, PDGFRA, PPP2R1A, STK11, MLL3, FOXL2, GNAS, HRAS, FGFR3, PTCH1, and CDH1.

14. The method of claim 12 wherein the level of the mutant nucleic acids and the total concentration of cell free nucleic acids are determined by Q-PCR.

15. The method of claim 12 wherein the level of the mutant nucleic acids is determined by amplifying a target nucleic acid sequence having less than 100 base pairs and which comprises the mutation of interest.

16. The method of claim 12 wherein the total concentration of cell free nucleic acids is determined by amplifying and quantifying a target acid nucleic sequence which has about the same size than the target nucleic acid sequence used for quantifying the mutant nucleic acid sequence.

17. The method according to claims 15 and 16 wherein the target nucleic sequence selected for determining the total concentration of cell free nucleic acids and the target nucleic acid sequence selected for determining the level of the mutant nucleic acids are located in the same gene.

18. The method according to claims 15 and 16 wherein the target nucleic sequence selected for determining the total concentration of cell free nucleic acids and the target nucleic acid sequence selected for determining the level of the mutant nucleic acids are located in the same exon of the same gene.

19. The method of claim 12 wherein for each mutation (M)n the ratio of step iv) (ML)n is determined and compared with its corresponding predetermined reference value (MLR)n and wherein the higher the number of (ML)n are higher than their corresponding predetermined values (MLR)n, the shorter will be the survival time of the patient.

20. A method for predicting the survival time of a patient suffering from a cancer comprising the steps of i) extracting the cell free nucleic acids from a sample obtained from the patient, ii) determining the level of the nucleic acids having a length inferior to 110 base pairs, iii) determining the level of the nucleic acids having a length superior to 250 base pairs, iv) calculating the ratio of the level determined at step iii) to the level determined at step ii), v) comparing the ratio determined at step iv) with a predetermined reference value and vi) concluding that the patient will a short survival time when the ratio determined at step iv) is lower than the predetermined reference value or concluding that the patient will have a long survival time when the level determined at step iv) is higher than the predetermined reference value.

21. The method of claim 20 wherein the level of the nucleic acids having a length inferior to 110 base pairs and the level of the nucleic acids having a length superior to 250 base pairs are determined by Q-PCR.

22. The method of claim 20 which consists of amplifying and quantifying a first target acid nucleic sequence having a length of inferior to 110 base pairs and a second target acid nucleic sequence having a length of at least 250 base pairs.

23. The method of claim 22 wherein the first target nucleic acid sequence has a length of 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; or 110 base pairs.

24. The method of claim 22 wherein the second target nucleic acid sequence has a length of 250; 251; 252; 253; 254; 255; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; 350 base pairs.

25. The method of claim 22 wherein the first and second target nucleic sequences are located in the same gene.

26. The method of claim 22 wherein the first and second target nucleic sequences are located in the same exon if the same gene.

27. The method of claim 22 wherein the first and second target nucleic sequences comprise a mutation of interest.

28. The method of claim 27 wherein the mutation of interest is located in a gene selected from the group consisting of KRAS, BRAF, NRAS, TP53, APC, MSH6, NF1, PIK3CA, SMAD4, EGFR, CDKN2A, IDH1, PTEN, SMARCB1, CTNNB1, HNF1A, VHL, ATM, EZH2, RET, NRAS, PTCH1, KIT, NF2, PDGFRA, PPP2R1A, STK11, MLL3, FOXL2, GNAS, HRAS, FGFR3, PTCH1, and CDH1.

29. The method of claim 27 which is performed for least 2 mutations, wherein for each mutation the ratio of step iv) is determined and compared with its corresponding predetermined reference value and wherein the higher the number of ratios are lower than their corresponding predetermined values, the shorter will be the survival time of the patient.

30. The method according to claim 1 which is combined with the determination of the total concentration of cell free nucleic acids present in the sample.

31. A method for predicting the survival time of a patient suffering from a cancer comprising the steps of i) extracting the cell free nucleic acids from a sample obtained from the patient, ii) determining the total concentration of cell free nucleic acids present in the sample, iii) determining the level of the nucleic acids having a length inferior to 110 base pairs, iv) determining the level of the nucleic acids having a length of superior to 250 base pairs, v) calculating the ratio of the level determined at step iv) to the level determined at step iii), vi) comparing the total concentration of cell free nucleic acids with its corresponding predetermined reference value, vii) comparing the ratio determined at step v) with its corresponding predetermined reference value and viii) concluding that the patient will a short survival time when
the total concentration determined at step i) is higher that its corresponding reference value and
the ratio determined at step v) is lower than its corresponding predetermined reference value.

32. The method for predicting the survival time of a patient suffering from a cancer according to claim 1 comprising the steps of the method according to claim 12.

33. The method for predicting the survival time of a patient suffering from a cancer according to claim 1 comprising the steps of the method according to claim 20.

34. The method for predicting the survival time of a patient suffering from a cancer according to claim 12 comprising the steps of the method according to claim 20.

35. The method for predicting the survival time of a patient suffering from a cancer according to claim 1, comprising the steps of the method according to claim 12 and the method according to claim 20.

36. The method of claim 35 which comprises the step consisting of i) extracting the cell free nucleic acids from a sample obtained from the patient, ii) determining the level of the mutant nucleic acids (as above described), iii) determining the total concentration of cell free nucleic acids present in the sample, iv) determining the mutation load, v) calculating the DNA integrity index, vi) comparing the level of the mutant nucleic acids with its corresponding predetermining reference value, vii) comparing the total concentration of cell free nucleic acids with its corresponding predetermined reference value viii) comparing the mutation load with its corresponding predetermined reference value, ix) comparing the DNA integrity index with is corresponding predetermined reference value and x) finally concluding that the patient will a short survival time when
the mutation is detected
the level of the mutant nucleic acids is higher than its corresponding predetermined reference value
the total concentration of cell free nucleic acids is higher than its corresponding predetermined reference value
the mutation load is higher than its corresponding predetermined reference value
the DNA integrity index is lower than its corresponding reference value.

37. The method according to claims 1, 12, 20 or 31 wherein the cancer is selected from the group consisting of neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

38. The method according to claims 1, 12, 20 or 31 wherein the patient suffers from a colorectal cancer.

39. The method according to claims 1, 12, 20 or 31 for predicting the duration of the overall survival (OS), progression-free survival (PFS) and/or the disease-free survival (DFS) of the cancer patient.

40. The method according to claims 1, 12, 20 or 31 for determining whether a patient is eligible or not to an anti-cancer treatment.

41. The method of claim 1, 12, 20 or 31 for determining whether a patient is eligible or not to an anti-cancer treatment wherein said anti-cancer treatment consists of radiotherapy, chemotherapy, immunotherapy or a combination thereof.

42. The method according to claim 1, 12, 20 or 31 wherein the patient suffers from a metastatic colorectal cancer.

43. The method of claim 12, wherein the mutation is a KRAS mutation or a BRAF mutation.

44. The method of claim 43 wherein the KRAS mutation is selected from the group consisting of G12C, G12D, G13D, G12R, and G12V or the BRAF mutation is V600E.

45. The method of claim 27, wherein the mutation is a KRAS mutation or a BRAF mutation.

46. The method of claim 45 wherein the KRAS mutation is selected from the group consisting of G12C, G12D, G13D, G12R, and G12V or the BRAF mutation is V600E.
